(19) United States
(12) Patent Application Publication
(10) Pub. No.: US 2025/0241523 A1
(43) Pub. Date: Jul. 31, 2025

(54) OBJECTIVE OPTICAL SYSTEM, ENDOSCOPE AND IMAGING DEVICE

(71) Applicant: OLYMPUS MEDICAL SYSTEMS CORP., Tokyo (JP)

(72) Inventor: Keisuke TAKADA, Kokubunji-shi (JP)

(73) Assignee: OLYMPUS MEDICAL SYSTEMS CORP., Tokyo (JP)

(21) Appl. No.: 19/182,747

(22) Filed: Apr. 18, 2025

Related U.S. Application Data
(63) Continuation of application No. PCT/JP2023/003781, filed on Feb. 6, 2023.

(51) Int. Cl. A61B 1/00

(52) U.S. Cl. CPC A61B 1/00188; A61B 1/00013

(57) ABSTRACT

An objective optical system is constituted by a first lens group with negative refractive power, a second lens group with positive refractive power, and a third lens group with positive refractive power, in order from an object side. The second lens group is moved to perform focusing. The first lens group is constituted by two lenses of a first lens that is a negative lens and a second lens that is a negative lens facing a concave surface on an image side. The third lens group has a single lens with positive refractive power and a bonded lens of a positive lens and a negative lens, in order from the object side. Provided that L1_Rr is a radius of curvature of an image side surface of the first lens, and L2_Rr is a radius of curvature of an image side surface of the second lens, 0.01<L1_Rr/L2_Rr<0.95.

### BACKGROUND OF THE INVENTION

### Field of the Invention

The present invention relates to an objective optical system, an endoscope and an imaging device. This application is a continuation application based on International Patent Application No. PCT/JP2023/003781 filed on Feb. 6, 2023, and the content of the PCT international application is incorporated herein by reference.

### Description of Related Art

Endoscopes are widely used as medical devices that allow users, such as engineers and doctors, to perform inspections, therapy, and treatments while directly viewing pictures of an affected area of a subject. Since an insertion part of the endoscope is inserted from outside a testee's body into the body, it is preferable that the overall length of an objective optical system attached to the insertion part be short and that a diameter of the objective optical system be small. In addition, in order to improve inspection accuracy of the affected area, etc., it is preferable to be able to image a small range with high resolution using the objective optical system. In the related art, an objective optical system that has a short overall length, small diameter and high resolution and can be installed on an endoscope has been proposed, and quality of the acquired images has been improved (for example, see Patent Documents 1 to 5).

### PATENT DOCUMENTS

[Patent Document 1] Japanese Patent No. 4819969

[Patent Document 2] Japanese Patent No. 5930257

[Patent Document 3] Japanese Patent No. 4819969

[Patent Document 4] Japanese Unexamined Patent Application, First Publication No. 2017-219783

[Patent Document 5] PCT International Patent Publication No. 2020/217443

### SUMMARY OF THE INVENTION

The objective optical systems disclosed in Patent Document 1 to Patent Document 5 can observe an object of an observation target by focusing on a far object point with a predetermined magnification and can enlarge and observe the object of the observation target by focusing on a near object point. For example, in the objective optical systems disclosed in Patent Document 1, Patent Document 3 and Patent Document 4, a first lens group to a third lens group are disposed in order from an object side to an image side, and only the second lens group moves when focusing. In the objective optical system disclosed in Patent Document 2, starting from the object side, a first lens with negative refractive power, a second meniscus lens, a third meniscus lens, a lens group with positive refractive power, and a bonded lens are disposed, and the third meniscus lens moves when focusing.

In the objective optical systems in the related art, including the objective optical systems disclosed in Patent Document 1 to Patent Document 5, a depth of field became shallower as the resolution increased and image quality improved when the systems were incorporated into imaging devices, and a sufficient depth of field could not be obtained. In addition, for example, in the objective optical systems disclosed in Patent Document 1 to Patent Document 4, the overall length and diameter of the optical system were not sufficiently shortened. In addition, for example, in the objective optical system disclosed in Patent Document 5, when trying to support auto focusing, there were cases where a space in which the lens group could move was insufficient.

In consideration of the above-mentioned problems, the present invention is directed to providing a small-diameter and high-performance objective optical system that has a focusing function, is capable of supporting auto-focusing, and with which a sufficient range in which movement is possible can be secured in a direction along a depth of field and an optical axis of a lens group. In addition, the present invention is directed to providing an endoscope and an imaging device each including the above-mentioned objective optical system.

An objective optical system of the present invention is constituted by a first lens group with negative refractive power, a second lens group with positive refractive power, and a third lens group with positive refractive power, in order from an object side. The second lens group is moved from the object side to an image side to perform focusing from a far object point to a near object point. The first lens group is constituted by two lenses of a first lens that is a negative lens and a second lens that is a negative lens facing a concave surface on the image side. The second lens group is constituted by a single positive meniscus lens facing a convex surface on the object side. The third lens group has a single lens with positive refractive power and a bonded lens of a positive lens and a negative lens, in order from the object side. The objective optical system of the present invention satisfies the following Conditional expression (1).

0.01<L1_Rr/L2_Rr<0.95 (1)
here, L1_Rr is a radius of curvature of an image side surface of the first lens, and L2_Rr is a radius of curvature of an image side surface of the second lens.

An endoscope of the present invention includes a tip portion in which the above-mentioned objective optical system is accommodated; an extension portion connected to a base end of the tip portion to be bendable; and an operation part connected to a base end opposite to a tip of the extension portion connected to the tip portion, and having a handle configured to change an axis shape of the extension portion.

An imaging device of the present invention includes the above-mentioned endoscope; and an imaging element configured to convert an image acquired by the objective optical system into an electrical signal.

According to the objective optical system of the present invention, since the second lens group with positive refractive power moves when focusing from the far object point to the near object point, it has a focusing function and can support auto focusing, ensuring a sufficient depth of field and movable region of the lens group, and achieving compactness. In addition, according to the objective optical system of the present invention, the first lens in the first lens group satisfies the above-mentioned conditional expression (1), which ensures the depth of field and achieves a small diameter, while at the same time achieving a balance between the aberrations that occur overall, achieving good correction, and achieving high performance. Further, according to the present invention, it is possible to provide the endoscope and the imaging device each including the above-mentioned objective optical system.

### BRIEF DESCRIPTION OF THE DRAWINGS

FIG. 1 is a cross-sectional view of an objective optical system of Example 1.

FIG. 2 is a cross-sectional view of an objective optical system of Example 2.

FIG. 3 is a cross-sectional view of an objective optical system of Example 3.

FIG. 4 is a cross-sectional view of an objective optical system of Example 4.

FIG. 5 is a cross-sectional view of an objective optical system of Example 5.

FIG. 6 is a cross-sectional view of an objective optical system of Example 6.

FIG. 7 is a cross-sectional view of an objective optical system of Example 7.

FIG. 8 is an aberration diagram of the objective optical system of Example 1.

FIG. 9 is an aberration diagram of the objective optical system of Example 2.

FIG. 10 is an aberration diagram of the objective optical system of Example 3.

FIG. 11 is an aberration diagram of the objective optical system of Example 4.

FIG. 12 is an aberration diagram of the objective optical system of Example 5.

FIG. 13 is an aberration diagram of the objective optical system of Example 6.

FIG. 14 is an aberration diagram of the objective optical system of Example 7.

FIG. 15 is a schematic view of an endoscope and an imaging device of an embodiment according to the present invention.

### DETAILED DESCRIPTION OF THE INVENTION

Hereinafter, an embodiment of an objective optical system, an endoscope and an imaging device according to an aspect of the present invention will be described with reference to the accompanying drawings. Further, while specific examples will be given when specifically describing the action and effect of this embodiment, as with the examples described later, the exemplified embodiment is a part of an aspect included in the present invention. In the exemplified embodiment, there are a plurality of variants. Accordingly, the present invention is not limited to the exemplified embodiment.

The objective optical system of the embodiment is, for example, incorporated into an endoscope and used to observe an affected area of a subject in the endoscope. In the objective optical system of the embodiment, it is possible to perform auto-focusing on a near object point, which is relatively close to the optical system, and a far object point, which is farther away than the near object point. By focusing on the near object point, the observation object can be observed at a magnification greater than a predetermined magnification. In addition, by focusing on the far object point, the observation object can be observed at the predetermined magnification.

The objective optical system of the embodiment is constituted by a first lens group with negative refractive power, a second lens group with positive refractive power, and a third lens group with positive refractive power. The first lens group, the second lens group, and the third lens group are disposed in order from an object side to an image side. As the second lens group moves from the object side to the image side, focusing from a far object point to a near object point is performed. Further, when focusing of the objective optical system of the embodiment, only the second lens group moves, and the first lens group and the third lens group are fixed.

In the objective optical system of the embodiment, the first lens group has a first lens that is a negative lens, and a second lens that is a negative lens with a concave surface facing the image side. The second lens group is constituted by a single positive meniscus lens with a convex surface facing the object side. The third lens group has a single lens with positive refractive power, and a bonded lens of a positive lens and a negative lens. In the third lens group, the single lens and the bonded lens are disposed in order from the object side.

Since the objective optical system of the embodiment has a plurality of lens groups from the first lens group to the third lens group, the diameter of the entire system in the plane perpendicular to the optical axis is restricted, and the second lens group is configured as a movable group. In addition, in the objective optical system of the embodiment, the first lens group has the first lens and the second lens that are negative lenses, and thus, a focal length of the entire system can be reduced and a depth of field can be secured. In addition, by providing the two negative lenses in the first lens group, color aberrations and coma aberrations can be corrected more effectively than when only one negative lens is used to shorten the focal length of the entire system. In other words, when the first lens group has only one negative lens, if the focal length of the entire system is intended to be shortened, color aberration and coma aberration occur significantly, making it impossible to obtain a high-performance objective optical system with good aberration performance.

In order to achieve a smaller diameter objective optical system while also ensuring a movable region of the second lens group when focusing, the movable region on the object side of the second lens group is required, and it is necessary to extend the maximum interval between an image side surface of the lens located closest to the image side of the first lens group and an object side surface of the lens located closest to the object side of the second lens group. The lenses and other optical elements in each lens group are held by a gripping member such as a telescope tube or the like from the outside in the radial direction within a plane perpendicular to the optical axis. For this reason, in the plane perpendicular to the optical axis and relatively outward in the radial direction, the air region on the object side of the second lens group, i.e., the maximum interval between the surface closest to the image side of the first lens group and the surface closest to the object side of the second lens group, must be secured. In addition, it is necessary to secure a space for disposing the third lens group and a back focus for adjusting the focus position.

Further, in this specification, the interval means an air gap, and means a spacing distance in the air between one surface and another surface in a direction parallel to the optical axis. Unless the context indicates otherwise, the interval means the spacing distance on the optical axis between one surface and the other surface in the objective optical system.

In the objective optical system of the embodiment, since the second lens group is constituted by positive meniscus lenses with their convex surfaces facing the object side, the principal points of the lenses that constitute the second lens group can be positioned on the object side of the second lens group. This makes it possible to secure the movable region of the second lens group in the objective optical system of the embodiment and the space on the object side of the second lens group, i.e., the space between the first lens group and the second lens group.

In order to achieve a shorter overall length of the objective optical system, it is preferable to have a small number of lenses that constitute the objective optical system. In the objective optical system of the embodiment, since the second lens group is constituted by the single positive meniscus lens, the length of the second lens group can be shortened, and as a result, the overall length can be shortened. Further, in this specification, the lens is an optical element, excluding a parallel plate, whose planes of the object side and the image side are parallel to a flat surface perpendicular to the optical axis, for example, a single lens or bonded lens. Either the object side surface or the image side surface of the lens includes a curved surface. The object side surfaces and the image side surfaces of the single lens and the bonded lens are in contact with the air.

In addition, in the objective optical system, it is preferable that a wider movable region is secured on the image side of the second lens group. However, for example, in the objective optical system including the three lens groups, when the space on the image side of the second lens group is simply reduced, aberrations that occur in the first and second lens groups may not be corrected and may remain on the imaging surface. Reducing the space on the image side of the second lens group means expanding the maximum interval between the image side surface of the lens located closest to the image side of the second lens group and the object side surface of the lens located closest to the object side of the third lens group.

The objective optical system of the embodiment has a third lens group including at least a single lens with positive refractive power and a bonded lens including a positive lens and a negative lens. For this reason, even when there are restrictions on the diameter and overall length of the entire system of the objective optical system in the embodiment and it is necessary to keep the diameter and overall length of the entire system within a predetermined range, aberrations can be corrected well, and spherical surface aberrations and color aberrations in particular can be well maintained. As a result, it is possible to obtain the objective optical system with good aberration performance.

In the objective optical system of the embodiment, in the third lens group, it is preferable that a lens with positive refractive power is disposed on the image side of the bonded lens with an interval therebetween. This lens acts as a cover glass that contacts the imaging surface from the object side. That is, the lens with positive refractive power may be arranged on the imaging surface of the imaging element, i.e., the object side surface, instead of the cover glass, which is a parallel plate. According to this configuration, alignment of the image plane of the objective optical system with respect to the imaging surface is performed by adjusting the interval between the bonded lens and the lens with positive refractive power disposed on the imaging surface of the imaging element. In addition, since the positive refractive power is exerted in a region on the image side of the objective optical system in the embodiment, that is, in a region close to the imaging surface, the error sensitivity of the alignment of the image plane is reduced. This makes it possible to suppress misalignment of the image plane during alignment of the image plane in the objective optical system of the embodiment.

In the objective optical system of the embodiment, an aperture diaphragm may be arranged in a space closer to the object side than the single lens arranged closest to the object side of the third lens group. However, regardless of the position of the aperture diaphragm parallel to the optical axis in the space between the second lens group and the third lens group, the image brightness and optical performance of the objective optical system do not change significantly. For this reason, the position of the aperture diaphragm disposed between the second lens group and the third lens group is determined according to the shape of each lens of the objective optical system or the telescope tube used to hold the aperture diaphragm. Further, the aperture diaphragm may be disposed in a space on the image side of the positive meniscus lens of the second lens group, and may be configured to move integrally with the second lens group while interlocking with the second lens group when focusing.

The objective optical system of the embodiment satisfies the following Conditional expression (1).

$$0.01 < L1\_Rr / L2\_Rr < 0 \text{.95} \qquad ( 1 )$$

However, in Conditional expression (1), L1_Rr is a radius of curvature of the image side surface of the first lens that is the negative lens of the first lens group, and L2_Rr is a radius of curvature of the image side surface of the second lens that is the negative lens of the first lens group.

Conditional expression (1) is conditional expression related to an appropriate ratio between the radius of curvature of the image side surface of the first lens and the radius of curvature of the image side surface of the second lens in the first lens group. The first lens is a single lens disposed closest to the object side in the plurality of single lens of the first lens group. The second lens is disposed on the image side of the first lens in the plurality of lenses of the first lens group, for example, in a space on the image side of the first lens.

In order to realize a retro-focus type configuration with the objective optical system of the embodiment, the first lens of the first lens group needs to have a relatively strong negative refractive power. However, if the negative refractive power of the first lens is made too strong, aberrations such as color aberration and coma aberration may become worse. Accordingly, in order to appropriately set the negative refractive power of the first lens, it is preferable to appropriately set the radius of curvature. In addition, adjusting only the negative refractive power of the first lens may not be sufficient to correct aberrations, making it difficult to realize an objective optical system with a deep depth of field. For this reason, it is necessary to provide negative refractive power to the second lens. Accordingly, in order to appropriately set the negative refractive power of the second lens, it is preferable to appropriately set the radius of curvature of each side surface of the first lens and the second lens.

By satisfying Conditional expression (1), in an objective optical system with a relatively small F number, such as the objective optical system in the embodiment, it is possible to achieve a good balance of aberration throughout the system and realize a compact objective optical system with a deep depth of field.

If it is lower than the lower limit value of Conditional Expression (1), the negative refractive power of the first lens in the first lens group becomes strong, which is undesirable since it makes color aberrations, coma aberrations, etc. more likely to occur. In addition, if it is lower than the lower limit value of Conditional expression (1), the negative refractive power of the second lens in the first lens group is not secured, and the depth of field of the objective optical system becomes shallow. As a result, this is not desirable because it increases the diameter of the first lens and the entire system, leading to an increase in the size of the objective optical system of the embodiment.

When it is higher than the upper limit value of Conditional expression (1), it is not preferable because the negative refractive power of the first lens in the first lens group cannot be secured and the depth of field of the objective optical system cannot be increased, and the diameter of the first lens is increased. In addition, when it is higher than the upper limit value of Conditional expression (1), the amount of aberration generated by the second lens in the first lens group becomes too large, coma aberration and magnification color aberration are particularly deteriorated, and it becomes difficult for the second lens group and third lens group, which are disposed after the first lens group, to correct the aberration worsened by the first lens group, as mentioned above.

It is preferable that the objective optical system of the embodiment satisfies the following Conditional expression (2).

$$0.136 < L 1 / L 2 < 0.95 \qquad ( 2 )$$

However, in Conditional expression (2), L1 indicates a focal length of the first lens of the first lens group, and L2 indicates a focal length of the second lens.

Conditional expression (2) is conditional expression related to an appropriate ratio between the negative refractive power of the first lens and the negative refractive power of the second lens of the first lens group. By satisfying Conditional expression (2), the aberrations of the objective optical system in the embodiment can be corrected satisfactorily, and a small-diameter objective optical system with a large depth of field can be realized.

When it is lower than the lower limit value of Conditional expression (2), it is not preferable because the negative refractive power of the first lens in the first lens group becomes stronger, the Petzval sum becomes larger, and the image plane curvature is over-corrected. In addition, when it is lower than the lower limit value of Conditional expression (2), since the negative refractive power of the second lens in the first lens group becomes too weak, the focal length of the entire system cannot be shortened, making it difficult to ensure the depth of field of the objective optical system of the embodiment.

When it is higher than the upper limit value of Conditional expression (2), the negative refractive power of the first lens in the first lens group becomes weaker, and the light height of the light incident on the first lens becomes larger, so that the diameter of the first lens becomes larger, which is undesirable. In addition, when it is higher than the upper limit value of Conditional expression (2), if the negative refractive power of the second lens in the first lens group becomes too strong, the position of the principal point moves to the image side, and as a result, the overall length of the objective optical system becomes longer, making it difficult to achieve a compact objective optical system of the embodiment.

It is preferable that the objective optical system of the embodiment satisfies the following Conditional expression (3).

$$0.2 < L2\_SF < 1 \text{.85} \qquad ( 3 )$$

However, in Conditional expression (3), L2_SF indicates a shaping factor of the second lens of the first lens group.

The shaping factor of the second lens of the first lens group is expressed by Equation (4) described below.

$$L2\_SF = ( L2\_Lr - L2\_Rr ) / ( L2\_Lr + L2\_Rr ) \qquad ( 4 )$$

In Equation (4), L2_Lr indicates a radius of curvature of the object side surface of the second lens of the first lens group, and L2_Rr indicates a radius of curvature of the image side surface of the second lens.

Conditional expression (3) is conditional expression related to a shape of the second lens of the first lens group. By satisfying Conditional expression (3), a small diameter of the objective optical system of the embodiment can be maintained, non-point aberration can be corrected well, and the objective optical system with a deep depth of field can be realized.

When it is lower than the lower limit value of Conditional expression (3), it is not preferable because the radius of curvature of the second lens in the first lens group becomes too large, making it impossible to maintain the negative refractive power of the second lens and resulting in a shallow depth of field.

When it is higher than the upper limit value of Conditional expression (3), the radius of curvature of the second lens in the first lens group becomes too small, non-point aberration worsens, making it difficult to ensure the aberration performance of the objective optical system of the embodiment.

It is preferable that the objective optical system of the embodiment satisfies the following Conditional expression (5).

$$- 0.4 < L 1 / f 2 < - 0 \text{.145} \qquad ( 5 )$$

However, in Conditional expression (5), L1 indicates a focal length of the first lens of the first lens group, and f2 indicates a focal length of the second lens group.

Conditional expression (5) relates to an appropriate ratio between the negative refractive power of the first lens of the first lens group and the positive refractive power of the second lens group. By satisfying Conditional expression (5), it is possible to effectively correct aberrations in the objective optical system and realize a compact objective optical system with a deep depth of field.

When it is lower than the lower limit value of Conditional expression (5), the negative refractive power of the first lens in the first lens group becomes weak, making it impossible to shorten the focal length and making it difficult to ensure the depth of field. In addition, when it is lower than the lower limit value of Conditional expression (5), the positive refractive power of the second lens group becomes too strong, deterioration in performance according to the eccentricity of the frame member holding the positive meniscus lens of the second lens group relative to the frame member holding the first lens will become significant, making it difficult to ensure the optical performance of the objective optical system when focusing.

When it is higher than the upper limit value of Conditional expression (5), the negative refractive power of the first lens in the first lens group becomes weak, and the Petzval sum becomes large, so that the image plane curvature tends to be overcorrected, which is no preferable. In addition, when it is higher than the upper limit value of Conditional expression (5), the positive refractive power of the second lens group becomes too weak, the error sensitivity according to the eccentricity of the frame member holding the positive meniscus lens relative to the frame member holding the first lens can be reduced, but the amount of movement of the second lens group becomes large, and the objective optical system of the embodiment becomes large, which is not preferable.

It is preferable that the objective optical system of the embodiment satisfies the following Conditional expression (6).

$$- 3 < L 1 / fw < - 0 \text{.955} \qquad ( 6 )$$

However, in Conditional expression (6), L1 indicates a focal length of the first lens of the first lens group, and fw indicates a focal length of the entire objective optical system of the embodiment when focusing to the far object point.

Conditional expression (6) relates to an appropriate ratio between the negative refractive power of the first lens of the first lens group and the refractive power of the entire objective optical system. By satisfying Conditional expression (6), it is possible to effectively correct aberrations in the objective optical system and realize a compact objective optical system with a deep depth of field.

When it is lower than the lower limit value of Conditional expression (6), the negative refractive power of the first lens in the first lens group becomes too weak, making it difficult to shorten the overall length of the objective optical system of the embodiment.

When it is higher than the upper limit value of Conditional expression (6), the negative refractive power of the first lens in the first lens group becomes too strong, coma aberrations and non-point aberrations tend to occur and become large, which is not preferable. In addition, when it is higher than the upper limit value of Conditional expression (6), since the radius of curvature of the image side surface of the first lens becomes too small, the error sensitivity according to the eccentricity with respect to the optical axis of the first lens tends to become large, which is not preferable.

It is preferable that the objective optical system of the embodiment satisfies the following Conditional expression (7).

$$- 3 < f 1 / fw < - 1 \text{.06} \qquad ( 7 )$$

However, in Conditional expression (7), f1 is a focal length of the first lens group, and fw is a focal length of the entire objective optical system of the embodiment when focusing to the far object point.

Conditional expression (7) relates to an appropriate ratio between the refractive power of the first lens group and the refractive power of the entire objective optical system. By satisfying Conditional expression (7), it is possible to effectively correct aberrations and achieve a compact objective optical system for the embodiment.

When it is lower than the lower limit value of Conditional expression (7), the negative refractive power of the first lens group becomes too weak, the light height of the light incident on the first lens of the first lens group becomes large, and the diameter of the first lens becomes large, which is not preferable.

When it is higher than the upper limit value of Conditional expression (7), the negative refractive power of the first lens group becomes too strong, the focal point of the first lens group approaches itself, i.e., the object side, and as a result, the overall length of the objective optical system of the embodiment becomes long, which is not preferable.

It is preferable that the objective optical system of the embodiment satisfies the following Conditional expression (8).

$$0.25 < thi\_ 3 g\_L1 / thi\_ 3 g\_air < 1.5 \qquad ( 8 )$$

However, in Conditional expression (8), thi_3g_L1 indicates a thickness on the optical axis of the single lens of the third lens group, and thi_3g_air indicates an air gap on the optical axis between the single lens and the bonded lens in the third lens group.

Conditional expression (8) is conditional expression related to a ratio between the thickness on the optical axis of the first single lens from the object side of the third lens group and the distance on the optical axis between the single lens and the bonded lens in the third lens group. By satisfying Conditional expression (8), it is possible to achieve a compact third lens group and achieve a compact objective optical system of the embodiment.

When it is lower than the lower limit value of Conditional expression (8), since the interval between the single lens and the bonded lens in the third lens group becomes large, the overall length of the objective optical system of the embodiment becomes long, which is not preferable.

When it is higher than the upper limit value of Conditional expression (8), since the single lens of the third lens group becomes large, the overall length of the objective optical system of the embodiment becomes long, which is not preferable.

It is preferable that the objective optical system of the embodiment satisfies the following Conditional expression (9).

$$0.325 < v / fw < 0.6 \qquad ( 9 )$$

However, in Conditional expression (9), v indicates an amount of movement of the second lens group from when focusing to the far object point to when focusing to the near object point, and fw indicates a focal length of the entire objective optical system of the embodiment when focusing to the far object point.

Conditional expression (9) is conditional expression related to an amount of movement on the optical axis of the positive meniscus lens of the second lens group. In order to achieve compactness and high performance in an objective optical system that has a movable group, such as the objective optical system in the embodiment, it is important to appropriately suppress the amount of movement of the movable group. By satisfying Conditional expression (9), the amount of movement on the optical axis of the second lens group, which is the movable group of the objective optical system of the embodiment, can be appropriately set according to the focal length of the entire objective optical system of the embodiment when focusing to the far object point, thereby achieving a compact size and high performance of the objective optical system of the embodiment.

When it is lower than the lower limit value of Conditional expression (9), this increases the error sensitivity of the image plane position in the objective optical system of the embodiment to the amount of movement of the positive meniscus lens of the second lens group, which is not preferable.

When it is higher than the upper limit value of Conditional expression (9), although the interval between the first lens group and the second lens group becomes larger and it is possible to ensure the amount of movement of the second lens group, the overall length of the objective optical system of the embodiment becomes longer, making it difficult to achieve a compact objective optical system of the embodiment.

It is preferable that the objective optical system of the embodiment satisfies the following Conditional expression (10).

$$- 0 \text{.4} < G3\_L1 \_SF < 0.4 \qquad ( 10 )$$

However, in Conditional expression (10), G3_L1_SF indicates a shaping factor of the single lens of the third lens group.

The shaping factor of the single lens of the third lens group is expressed by Equation (11) described below.

$$G3\_L1 \_SF = ( G3\_L1 \_Lr + G3\_L1 \_Rr ) / G3\_L1 \_Lr - G 3 \_L1 \_Rr ) \qquad ( 11 )$$

In Euqation (11), G3_L1_Lr indicates a radius of curvature of the object side surface of the single lens of the third lens group, and G3_L1_Rr indicates a radius of curvature of the image side surface of the single lens.

Conditional expression (10) is conditional expression related to a shape of the single lens of the third lens group. By satisfying Conditional expression (10), it is possible to achieve a compact optical system of the embodiment and to perform good correction of spherical surface aberrations and coma aberrations.

When it is lower than the lower limit value of Conditional expression (10), the radius of curvature of either the object side surface or the image side surface of the single lens in the third lens group becomes too small, making it difficult to correct spherical surface aberrations and coma aberrations.

Even when it is higher than the upper limit value of Conditional expression (10), the radius of curvature of either the object side surface or the image side surface of the single lens in the third lens group becomes too small, making it difficult to correct spherical surface aberrations and coma aberrations.

It is preferable that the objective optical system of the embodiment satisfies the following Conditional expression (12).

$$- 1 \text{.5} < G3\_Lce \_SF < - 0.2 \qquad ( 12 )$$

However, in Conditional expression (12), G3_Lce_SF indicates a shaping factor of the bonded lens of the third lens group.

The shaping factor of the bonded lens of the third lens group is expressed by Equation (13) described below.

$$( 13 )$$

$$G3\_Lce \_SF = ( G3\_Lce \_Lr + G3\_Lce \_Rr ) / ( G3\_Lce \_Lr - G3\_Lce \_Rr )$$

In Equation (13), G3_Lce_Lr indicates a radius of curvature of the object side surface of the bonded lens in the third lens group, i.e., a radius of curvature of the object side surface of the lens disposed on the object side of the bonded lens and having the positive refractive power. In Equation (13), G3_Lce_Rr indicates a radius of curvature of the image side surface of the bonded lens of the third lens group, i.e., a radius of curvature of the image side surface of the lens disposed on the image side of the bonded lens and having the negative refractive power.

Conditional expression (12) is conditional expression related to a shape of the bonded lens in the third lens group. By satisfying Conditional expression (12), it is possible to satisfactorily correct non-point aberrations and coma aberrations in the objective optical system of the embodiment.

When it is lower than the lower limit value of Conditional expression (12), the radius of curvature of the object side surface of the bonded lens in the third lens group becomes too small, which is not preferable since it makes coma aberrations more likely to occur. In addition, when it is lower than the lower limit value of Conditional expression (12), this is not preferable because it does not provide sufficient correction for coma aberrations and non-point aberrations caused by the image side surface of the bonded lens.

When it is higher than the upper limit value of Conditional expression (12), the radius of curvature of the object side surface of the bonded lens in the third lens group becomes too large, the refractive power of the bonded lens cannot be maintained and the overall length of the objective optical system in the embodiment becomes large, which is not preferable. In addition, when it is higher than the upper limit value of Conditional expression (12), the radius of curvature of the image side surface of the bonded lens becomes too small, which makes coma aberrations and non-point aberrations prone to over-correction, which is not preferable.

An endoscope of the embodiment includes a tip portion in which the objective optical system of the embodiment is accommodated, a bendable extension portion connected to a base end of the tip portion, and an operation part connected to the base end opposite to a tip connected to the tip portion in the extension portion and having a handle configured to freely change an axis shape of the extension portion.

An imaging device of the embodiment includes the endoscope of the embodiment, and an imaging element configured to convert an image acquired by the objective optical system of the embodiment into an electrical signal.

An endoscope and an imaging device are provided that are compatible with auto focusing, can ensure sufficient depth of field and movable region, and is equipped with a small-diameter and high-performance objective optical system, allowing for simple operation to observe an observation object with high accuracy and diagnose the affected area of the observation object, etc.

For the above-mentioned Conditional expressions (1) to (3), (5) to (10), and (12), at least one of the lower limit value and the upper limit value may be changed as follows. By making such changes, the effect of satisfying each conditional expression becomes stronger.

Conditional expression (1) is as follows.

It is more preferable to set the lower limit value at 0.1, and even more preferable to set it at 0.335.

It is more preferable to set the upper limit value at 0.9, and even more preferable to set it at 0.795.

Conditional expression (2) is as follows.

It is more preferable to set the lower limit value at 0.2, and even more preferable to set it at 0.25.

It is more preferable to set the upper limit value at 0.8, and even more preferable to set it at 0.625.

Conditional expression (3) is as follows.

It is more preferable to set the lower limit value at 0.3, and even more preferable to set it at 0.55.

It is more preferable to set the upper limit value at 1.7, and even more preferable to set it at 1.5.

Conditional expression (5) is as follows.

It is more preferable to set the lower limit value at −0.35, and even more preferable to set it at −0.25.

It is more preferable to set the upper limit value at −0.15, and even more preferable to set it at −0.16.

Conditional expression (6) is as follows.

It is more preferable to set the lower limit value at −2.8, and even more preferable to set it at −2.5.

It is more preferable to set the upper limit value at −1.0, and even more preferable to set it at −1.5.

Conditional expression (7) is as follows.

It is more preferable to set the lower limit value at −2.0, and even more preferable to set it at −1.6.

It is more preferable to set the upper limit value at −1.1, and even more preferable to set it at −1.2.

Conditional expression (8) is as follows.

It is more preferable to set the lower limit value at 0.3, and even more preferable to set it at 0.4.

It is more preferable to set the upper limit value at 1.4, and even more preferable to set it at 1.25.

Conditional expression (9) is as follows.

It is more preferable to set the lower limit value at 0.35.

It is more preferable to set the upper limit value at 0.55.

Conditional expression (10) is as follows.

It is more preferable to set the lower limit value at −0.35, and even more preferable to set it at −0.3.

It is more preferable to set the upper limit value at 0.3, and even more preferable to set it at 0.25.

Conditional expression (12) is as follows.

It is more preferable to set the lower limit value at −1.4, and even more preferable to set it at −1.3.

It is more preferable to set the upper limit value at −0.3, and even more preferable to set it at −0.35.

Next, examples of the objective optical system of the embodiment will be described. Further, the present invention is not limited to the following examples.

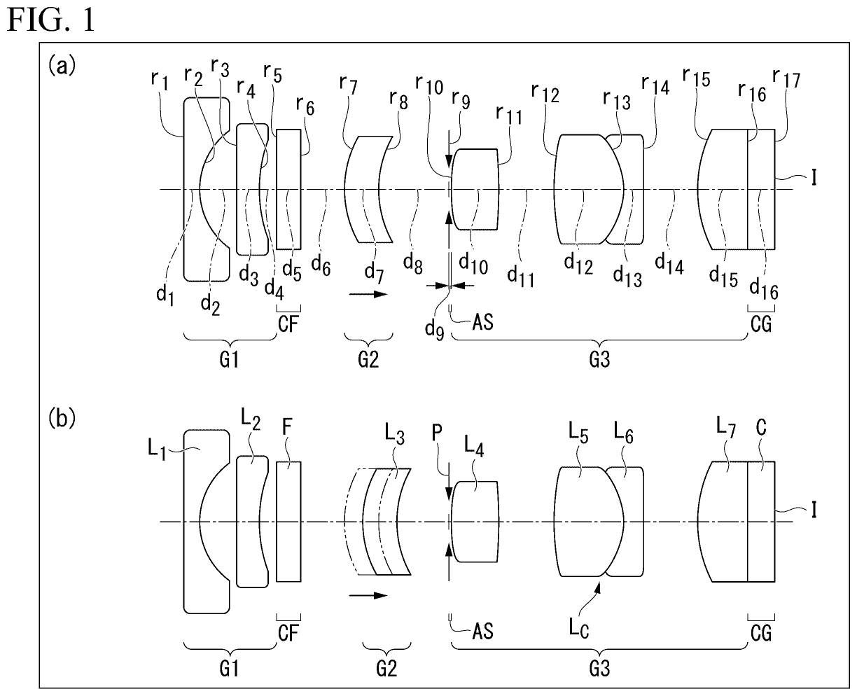

FIG. 1 to FIG. 7 are cross-sectional views of objective optical systems of Example 1 to Example 7. In the drawings of FIG. 1 to FIG. 7, (a) is a cross-sectional view when focusing to the far object point, and (b) is a cross-sectional view when focusing to the near object point. In (b) of each of the drawings of FIG. 1 to FIG. 7, for reference, the position of the meniscus lens of the second lens group when focusing to the far object point is shown by a two-dot chain line.

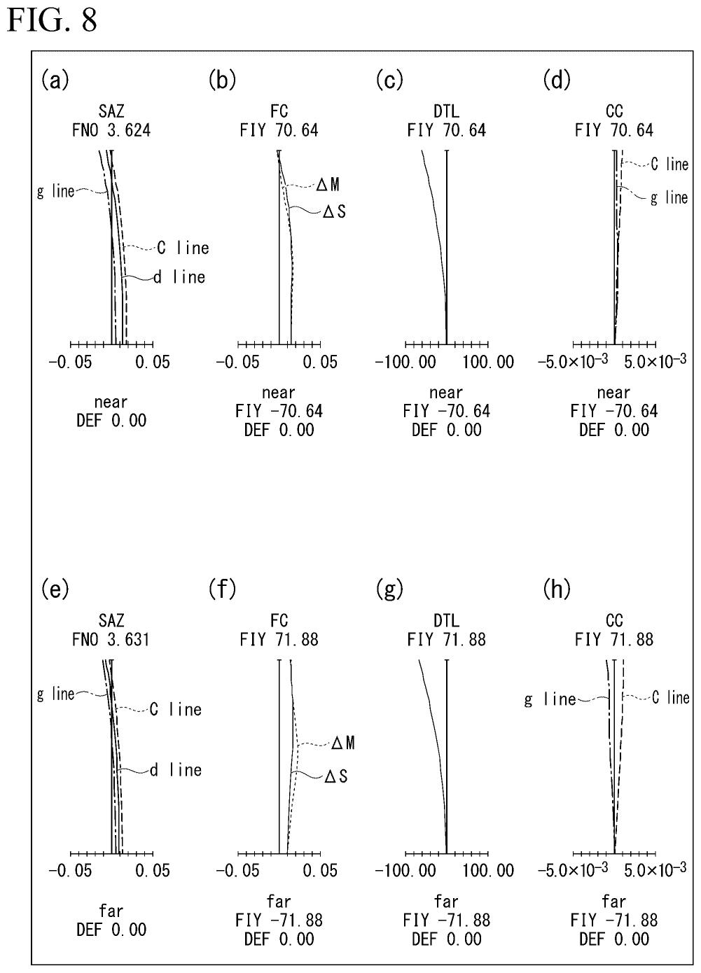

FIG. 8 to FIG. 14 are aberration diagrams of the objective optical systems of Example 1 to Example 7. In each of the drawings of FIG. 1 to FIG. 7, (a), (b), (c) and (d) are aberration diagram when focusing to the far object point, and (e), (f), (g) and (h) are aberration diagram when focusing to the near object point. In each of the drawings of FIG. 1 to FIG. 7, (a) and (e) are views of spherical surface aberration (spherical aberration; SA). Here, (b) and (f) are views of non-point aberration (astigmatism; AS). Here, (c) and (g) are views of distortion aberration (distortion; DT). Here, (d) and (h) are views of magnification color aberration (lateral chromatic aberration; CC). In each of (a), (d), (e) and (h), line g expresses each aberration at a wavelength of 435.84 nm, line C expresses each aberration at a wavelength of 656.27 nm. In (a) and (e), line d expresses each aberration at a wavelength of 587.56 nm. In (b) and (f), ΔM expresses aberration with respect to a meridional image plane in line d, and ΔS expresses aberration with respect to a sagittal image plane in line d.

In FIG. 1 to FIG. 7, a first lens group of the objective optical system in each example is designated by G1, a second lens group is designated by G2, a third lens group is designated by G3, an infrared filter is designated by CF, an aperture diaphragm is designated by AS, a cover glass is designated by CG, and an image plane, i.e., an imaging surface is designated by I.

As shown in FIG. 1 to FIG. 7, each of the objective optical systems of Example 1 to Example 7 has the first lens group G1 with negative refractive power, the second lens group G2 with positive refractive power, and the third lens group G3 with positive refractive power in order from the object side.

In each of the objective optical systems of Example 1 to Example 7, when focusing from the far object point to the near object point, the second lens group G2 is moved from the object side to the image side, and the first lens group G1 and the third lens group G3 are fixed. Here, the infrared filter F is fixed like the first lens group G1. The aperture diaphragm P is interlocked with the second lens group G2 to move on the optical axis. The cover glass C is fixed like the third lens group G3.

In each of the objective optical systems of Example 1 to Example 7, the non-spherical surface is provided on an object side surface of a meniscus lens L3 of the second lens group G2.

Hereinafter, a specific configuration of each of the objective optical systems of Example 1 to Example 7 will be described.

### Example 1

As shown in FIG. 1, in the objective optical system of Example 1, the first lens group G1 is constituted by a plano-concave lens L₁ with negative refractive power, and a plano-concave lens L₂ with negative refractive power, in order from the object side. The plano-concave lens L₁ corresponds to “a first lens” disclosed in the following claims. The plano-concave lens L₂ corresponds to “a second lens” disclosed in the following claims. The plano-concave lenses L₁ and L₂ face a concave surface on the image side. That is, the object side surface of the plano-concave lenses L₁ and L₂ is a flat surface perpendicular to the optical axis. The image side surface of the plano-concave lenses L₁ and L₂ is a concave surface recessed to the object side. The infrared filter F is disposed in the first lens group G1, and specifically, disposed in a space on the image side of the plano-concave lens L₂. The object side surface and the image side surface of the infrared filter F are flat surfaces perpendicular to the optical axis.

The second lens group G2 is constituted by the meniscus lens L₃ that is a single positive meniscus lens. The meniscus lens L₃ corresponds to “a positive meniscus lens” disclosed in the following claims. The meniscus lens L₃ faces a convex surface on the object side. That is, the object side surface and the image side surface of the meniscus lens L₃ are concave surfaces recessed to the object side.

The third lens group G3 is constituted by a biconvex lens L₄ with positive refractive power, a biconvex lens L₅ that is a positive lens, a meniscus lens L₆ that is a negative meniscus lens, and a plano-convex lens L₇ with positive refractive power, in order from the object side. The aperture diaphragm P is disposed in the third lens group G3, and specifically, a space on the object side of the biconvex lens L₄. An opening having a diameter smaller than that of the biconvex lens L₄ is formed in the aperture diaphragm P about the optical axis. The biconvex lens L₄ corresponds to “a single lens” disclosed in the following claims. The object side surfaces of the biconvex lenses L₄ and L₅ are convex surfaces protruding to the object side. The image side surfaces of the biconvex lenses L₄ and L₅ are convex surfaces protruding to the image side. The object side surface of the meniscus lens L₆ is a concave surface recessed to the image side. The image side surface of the meniscus lens L₆ is a convex surface protruding to the image side. The biconvex lens L₅ and the meniscus lens L₆ are joined to each other to constitute a single bonded lens L_{C}. That is, the image side surface of the biconvex lens L₅ and the object side surface of the meniscus lens L₆ are in contact with each other. The bonded lens L_{C} corresponds to “a bonded lens” disclosed in the following claims.

The object side surface of the plano-convex lens L₇ is a convex surface protruding to the object side. The image side surface of the plano-convex lens L₇ is a flat surface perpendicular to the optical axis. The cover glass C is disposed in a space on the image side of the third lens group G3. The object side surface and the image side surface of the cover glass C are flat surfaces perpendicular to the optical axis. The image side surface of the cover glass C is an image plane I of the objective optical system, i.e., an imaging surface. The image side surface of the plano-convex lens L₇ and the object side surface of the cover glass C are in contact with each other.

Table 1 to Table 5 show numerical data of Example 1. Further, common to each of the numerical data in Examples 1 to 7, in the surface data, r is a radius of curvature of each surface, d is an interval between the surfaces, nd is a wavelength of each lens (587.56 nm), i.e., a refractive index at the line d, and vd is an Abbe number of each lens. A unit of each numerical value is in millimeters [mm]. A sign * indicates a non-spherical surface. AS indicates an aperture diaphragm.

In addition, common to each of the numerical data in Examples 1 to 7, the non-spherical surface shape is expressed by the following equation, where a direction parallel to the optical axis of the objective optical system is z, a direction perpendicular to the optical axis is y, a conic coefficient is k, and the non-spherical surface coefficients are A4, A6, A8, and A10.

$$z = ( {y}^{2} / r ) / [ 1 + {\{ 1 - ( 1 + k ) {( y / r )}^{2} \}}^{1 / 2} ] + A 4 {y}^{4} + A 6 {y}^{6} + A 8 {y}^{8} + A 10 {y}^{10}$$

### [Numerical Example 1]

TABLE 1
SURFACE DATA

| SURFACE NUMBER | r | d | nd | νd |
|---|---|---|---|---|
| 1 | ∞ | 0.2000 | 1.88300 | 40.76 |
| 2 | 0.961 | 0.4750 | | |
| 3 | ∞ | 0.3000 | 1.48749 | 70.23 |
| 4 | 1.961 | 0.2300 | | |
| 5 | ∞ | 0.3000 | 1.52100 | 65.12 |
| 6 | ∞ | D6 | | |
| 7* | 1.3376 | 0.4500 | 1.80610 | 40.92 |
| 8 | 1.534 | D8 | | |
| 9 | AS | 0.0300 | | |
| 10 | 3.320 | 0.6000 | 1.88300 | 40.76 |
| 11 | −4.950 | 0.7250 | | |
| 12 | 2.920 | 0.9000 | 1.83481 | 42.71 |
| 13 | −1.020 | 0.2700 | 1.92286 | 18.90 |
| 14 | −13.387 | 0.7000 | | |
| 15 | 1.697 | 0.6500 | 1.51633 | 64.14 |
| 16 | ∞ | 0.3500 | 1.51000 | 62.01 |
| 17 | ∞ | 0.0000 | | |

TABLE 2
NON-SPHERICAL DATA

| SURFACE | | |
|---|---|---|
| NUMBER | R | k |
| 7 | 1.3376 | 0.0000 |

| A4 | A6 | A8 | A10 |
|---|---|---|---|
| −3.97474 × 10⁻² | 1.44684 × 10⁻¹ | 1.14746 × 10⁻¹ | −1.59678 × 10⁰ |

TABLE 3
ZOOM DATA

| | FAR OBJECT | NEAR OBJECT |
|---|---|---|
| | POINT | POINT |
| OBJECT DISTANCE | 10.500 | 2.850 |
| FOCAL LENGTH | 0.5131 | 0.5138 |
| F NUMBER | 3.631 | 3.624 |
| ANGLE OF VIEW (2ω) | 144.11 | 141.60 |
| IMAGE HEIGHT | 0.500 | 0.500 |
| OVERALL LENGTH | 7.680 | 7.680 |
| D6 | 0.5800 | 0.8100 |
| D8 | 0.9200 | 0.6900 |

TABLE 4
FOCAL LENGTH OF EACH LENS GROUP

| | |
|---|---|
| FIRST LENS GROUP | −0.7521 |
| SECOND LENS GROUP | 6.3591 |
| THIRD LENS GROUP | 1.8713 |

TABLE 5
CORRESPONDING VALUES OF
CONDITIONAL EXPRESSIONS

| | | |
|---|---|---|
| CONDITIONAL EXPRESSION (1) | L1_Rr/L2_Rr | 0.490 |
| CONDITIONAL EXPRESSION (2) | L1/L2 | 0.270 |
| CONDITIONAL EXPRESSION (3) | L2_SF | 1.000 |
| CONDITIONAL EXPRESSION (5) | L1/f2 | −0.170 |
| CONDITIONAL EXPRESSION (6) | L1/fw | −2.109 |
| CONDITIONAL EXPRESSION (7) | f1/fw | −1.466 |
| CONDITIONAL EXPRESSION (8) | thi_3g_L1/thi_3g_air | 0.828 |
| CONDITIONAL EXPRESSION (9) | v/fw | 0.448 |
| CONDITIONAL EXPRESSION (10) | G3_L1_SF | −0.197 |
| CONDITIONAL EXPRESSION (12) | G3_Lce_SF | −0.642 |

As can be seen from FIG. 8, the objective optical system of Example 1 corrects aberrations, for example, spherical surface aberrations, non-point aberrations, distortion aberrations and magnification color aberrations, and provides good aberration characteristics over the visible wavelength range.

### Example 2

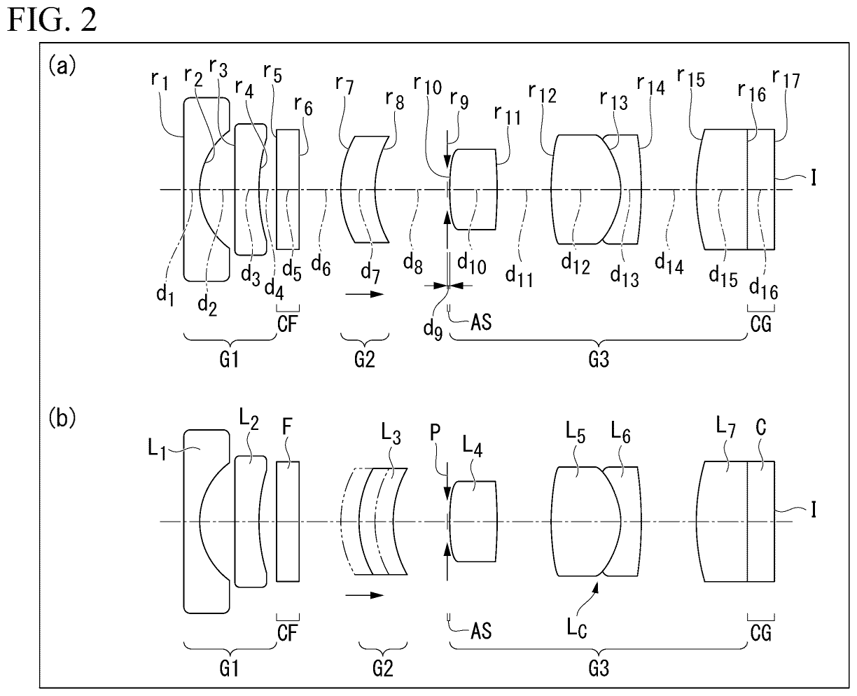

As shown in FIG. 2, in Example 2, the first lens group G1 is constituted by the plano-concave lens L₁ with negative refractive power and the plano-concave lens L₂ with negative refractive power, in order from the object side. Further, in each of the examples after Example 2, lenses of the same type as those in the previously explained examples are given the same reference signs as those in the previously explained examples, and descriptions of the object side surfaces and the image planes of those lenses, etc. will be omitted. The infrared filter F is disposed in a space on the image side of the plano-concave lens L₂.

The second lens group G2 is constituted by the single meniscus lens L₃ that is a positive meniscus lens.

The third lens group G3 is constituted by the biconvex lens L₄ with positive refractive power, the biconvex lens L₅ that is a positive lens, the meniscus lens L₆ that is a negative meniscus lens, and the plano-convex lens L₇ with positive refractive power, in order from the object side. The aperture diaphragm P is disposed in a space on the object side of the biconvex lens L₄. The biconvex lens L₅ and the meniscus lens L₆ constitute the bonded lens L_{C}. The cover glass C is disposed in a space on the image side of the third lens group G3.

Table 6 to Table 10 show numerical data of Example 2.

### [Numerical Example 2]

TABLE 6
SURFACE DATA

| SURFACE NUMBER | r | d | nd | νd |
|---|---|---|---|---|
| 1 | ∞ | 0.2000 | 1.88300 | 40.76 |
| 2 | 0.961 | 0.4750 | | |
| 3 | ∞ | 0.3000 | 1.51633 | 64.14 |
| 4 | 2.124 | 0.2200 | | |
| 5 | ∞ | 0.3000 | 1.53962 | 62.47 |
| 6 | ∞ | D6 | | |
| 7* | 1.3321 | 0.4500 | 1.80625 | 40.91 |
| 8 | 1.529 | D8 | | |
| 9 | AS | 0.0300 | | |
| 10 | 3.071 | 0.6000 | 1.88300 | 40.76 |
| 11 | −5.302 | 0.7200 | | |
| 12 | 2.722 | 0.9000 | 1.77250 | 49.60 |
| 13 | −1.020 | 0.2700 | 1.92286 | 18.90 |
| 14 | −5.875 | 0.7300 | | |
| 15 | 2.960 | 0.6600 | 1.88300 | 40.76 |
| 16 | ∞ | 0.3500 | 1.51000 | 62.01 |
| 17 | ∞ | 0.0000 | | |

TABLE 7
NON-SPHERICAL DATA

| SURFACE | | |
|---|---|---|
| NUMBER | R | k |
| 7 | 1.3321 | 0.0000 |

| A4 | A6 | A8 | A10 |
|---|---|---|---|
| −4.22247 × 10⁻² | 9.24108 × 10⁻² | 3.80047 × 10⁻¹ | −1.93899 × 10⁰ |

TABLE 8
ZOOM DATA

| | FAR OBJECT | NEAR OBJECT |
|---|---|---|
| | POINT | POINT |
| OBJECT DISTANCE | 10.500 | 3.000 |
| FOCAL LENGTH | 0.5148 | 0.5163 |
| F NUMBER | 3.678 | 3.674 |
| ANGLE OF VIEW (2ω) | 143.10 | 140.32 |
| IMAGE HEIGHT | 0.500 | 0.500 |
| OVERALL LENGTH | 7.705 | 7.705 |
| D6 | 0.5550 | 0.7850 |
| D8 | 0.9450 | 0.7150 |

TABLE 9
FOCAL LENGTH OF EACH LENS GROUP

| | |
|---|---|
| FIRST LENS GROUP | −0.7577 |
| SECOND LENS GROUP | 6.3019 |
| THIRD LENS GROUP | 1.8942 |

TABLE 10
CORRESPONDING VALUES OF
CONDITIONAL EXPRESSIONS

| | | |
|---|---|---|
| CONDITIONAL EXPRESSION (1) | L1_Rr /L2_Rr | 0.452 |
| CONDITIONAL EXPRESSION (2) | L1/L2 | 0.264 |
| CONDITIONAL EXPRESSION (3) | L2_SF | 1.000 |
| CONDITIONAL EXPRESSION (5) | L1/f2 | −0.172 |
| CONDITIONAL EXPRESSION (6) | L1/fw | −2.102 |
| CONDITIONAL EXPRESSION (7) | f1/fw | −1.472 |
| CONDITIONAL EXPRESSION (8) | thi_3g_L1/thi_3g_air | 0.833 |
| CONDITIONAL EXPRESSION (9) | v/fw | 0.447 |
| CONDITIONAL EXPRESSION (10) | G3_L1_SF | −0.266 |
| CONDITIONAL EXPRESSION (12) | G3_Lce_SF | −0.367 |

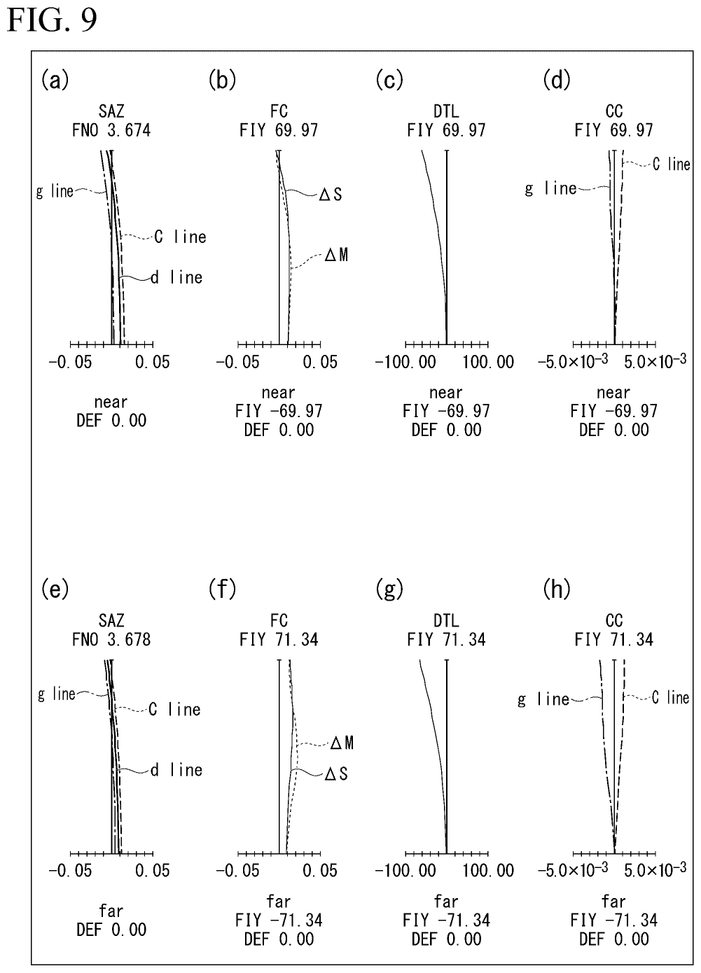

As can be seen from FIG. 9, the objective optical system of Example 2 also corrects aberrations, for example, spherical surface aberration, non-point aberration, distortion aberration and magnification color aberration, and provides good aberration characteristics in the visible wavelength range.

### Example 3

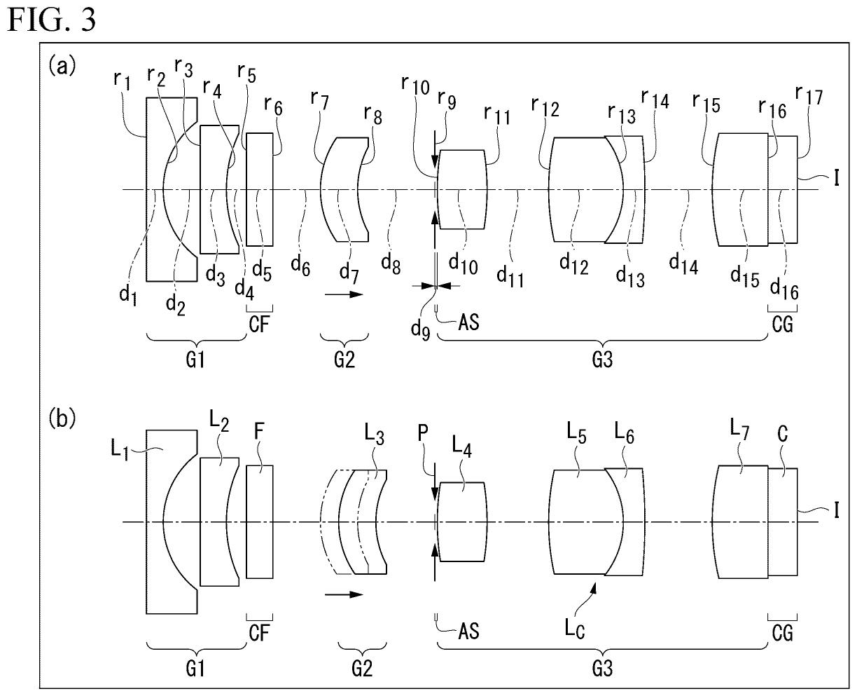

As shown in FIG. 3, in Example 3, the first lens group G1 is constituted by the plano-concave lens L₁ with negative refractive power and the plano-concave lens L₂ with negative refractive power, in order from the object side. The infrared filter F is disposed in a space on the image side of the plano-concave lens L₂.

The second lens group G2 is constituted by the single meniscus lens L₃ that is a positive meniscus lens.

The third lens group G3 is constituted by the biconvex lens L₄ with positive refractive power, the biconvex lens L₅ that is a positive lens, the meniscus lens L₆ that is a negative meniscus lens, and the plano-convex lens L₇ with positive refractive power, in order from the object side. The aperture diaphragm P is disposed in a space on the object side of the biconvex lens L₄. The biconvex lens L₅ and the meniscus lens L₆ constitute the bonded lens L_{C}. The cover glass C is disposed in a space on the image side of the third lens group G3.

Table 11 to Table 15 show numerical data of Example 3.

### [Numerical Example 3]

TABLE 11
SURFACE DATA

| SURFACE NUMBER | r | d | nd | νd |
|---|---|---|---|---|
| 1 | ∞ | 0.2000 | 1.88300 | 40.76 |
| 2 | 0.999 | 0.4582 | | |
| 3 | ∞ | 0.3000 | 1.51633 | 64.14 |
| 4 | 1.572 | 0.2506 | | |
| 5 | ∞ | 0.3000 | 1.52100 | 65.12 |
| 6 | ∞ | D6 | | |
| 7* | 1.0433 | 0.4500 | 1.80610 | 40.92 |
| 8 | 1.077 | D8 | | |
| 9 | AS | 0.0300 | | |
| 10 | 2.895 | 0.6000 | 1.80100 | 34.97 |
| 11 | −4.178 | 0.7345 | | |
| 12 | 2.726 | 0.9000 | 1.81600 | 46.62 |
| 13 | −1.020 | 0.2700 | 1.95906 | 17.47 |
| 14 | −7.912 | 0.8125 | | |
| 15 | 2.960 | 0.6600 | 1.88300 | 40.76 |
| 16 | ∞ | 0.3500 | 1.51000 | 62.01 |
| 17 | ∞ | 0.0000 | | |

TABLE 12
NON-SPHERICAL DATA

| SURFACE | | |
|---|---|---|
| NUMBER | R | k |
| 7 | 1.0433 | 0.0000 |

| A4 | A6 | A8 | A10 |
|---|---|---|---|
| −5.27109 × 10⁻² | −5.06765 × 10⁻² | 7.03624 × 10⁻¹ | −2.19506 × 10⁰ |

TABLE 13
ZOOM DATA

| | FAR OBJECT | NEAR OBJECT |
|---|---|---|
| | POINT | POINT |
| OBJECT DISTANCE | 10.500 | 3.000 |
| FOCAL LENGTH | 0.5140 | 0.5192 |
| F NUMBER | 3.595 | 3.592 |
| ANGLE OF VIEW (2ω) | 143.52 | 138.40 |
| IMAGE HEIGHT | 0.500 | 0.500 |
| OVERALL LENGTH | 7.816 | 7.816 |
| D6 | 0.5800 | 0.8000 |
| D8 | 0.9200 | 0.7000 |

TABLE 14
FOCAL LENGTH OF EACH LENS GROUP

| | |
|---|---|
| FIRST LENS GROUP | −0.7090 |
| SECOND LENS GROUP | 5.8841 |
| THIRD LENS GROUP | 1.9368 |

TABLE 15
CORRESPONDING VALUES OF
CONDITIONAL EXPRESSIONS

| | | |
|---|---|---|
| CONDITIONAL EXPRESSION (1) | L1_Rr/L2_Rr | 0.636 |
| CONDITIONAL EXPRESSION (2) | L1/L2 | 0.371 |
| CONDITIONAL EXPRESSION (3) | L2_SF | 1.000 |
| CONDITIONAL EXPRESSION (5) | L1/f2 | −0.191 |
| CONDITIONAL EXPRESSION (6) | L1/fw | −2.189 |
| CONDITIONAL EXPRESSION (7) | f1/fw | −1.379 |
| CONDITIONAL EXPRESSION (8) | thi_3g_L1/thi_3g_air | 0.817 |
| CONDITIONAL EXPRESSION (9) | v/fw | 0.428 |
| CONDITIONAL EXPRESSION (10) | G3_L1_SF | −0.181 |
| CONDITIONAL EXPRESSION (12) | G3_Lce_SF | −0.487 |

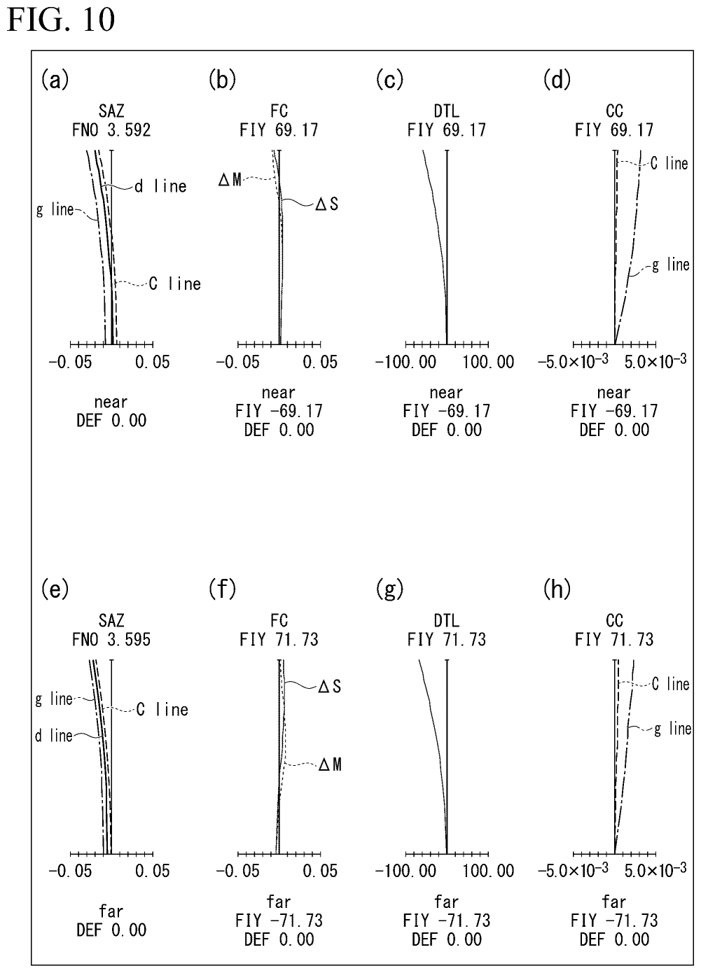

As can be seen from FIG. 10, the objective optical system of Example 3 also corrects aberrations, for example, spherical surface aberrations, non-point aberrations, distortion aberrations and magnification color aberrations, and provides good aberration characteristics in the visible wavelength range.

### Example 4

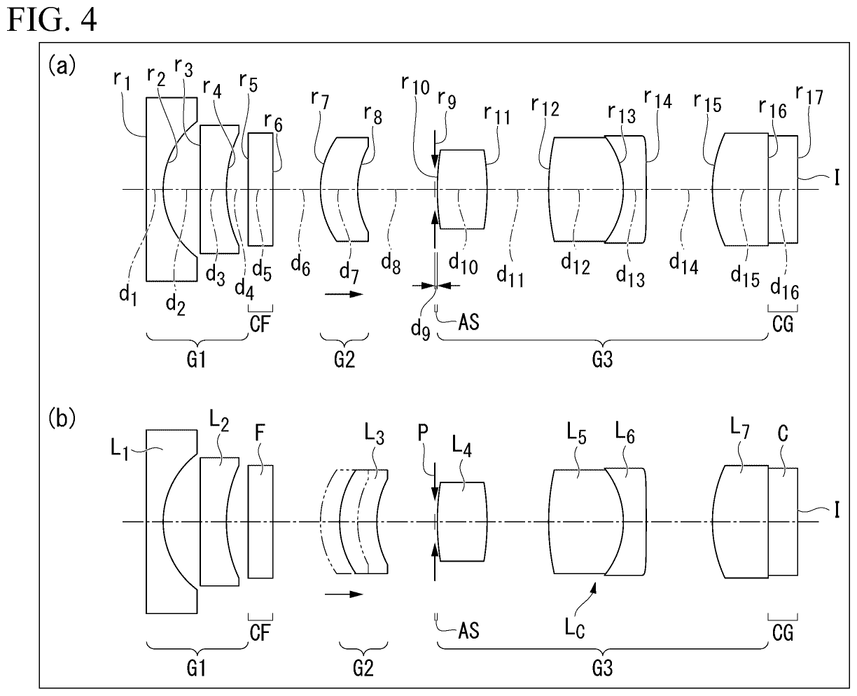

As shown in FIG. 4, in Example 4, the first lens group G1 is constituted by the plano-concave lens L₁ with negative refractive power and the plano-concave lens L₂ with negative refractive power, in order from the object side. The infrared filter F is disposed in a space on the image side of the plano-concave lens L₂.

The second lens group G2 is constituted by the single meniscus lens L₃ that is a positive meniscus lens.

The third lens group G3 is constituted by the biconvex lens L₄ with positive refractive power, the biconvex lens L₅ that is a positive lens, the meniscus lens L₆ that is a negative meniscus lens, and the plano-convex lens L₇ with positive refractive power, in order from the object side. The aperture diaphragm P is disposed in a space on the object side of the biconvex lens L_{4.} The biconvex lens L₅ and the meniscus lens L₆ constitute the bonded lens L_{C}. The cover glass C is disposed in a space on the image side of the third lens group G3.

Table 16 to Table 20 show numerical data of Example 4.

### [Numerical Example 4]

TABLE 16
SURFACE DATA

| SURFACE NUMBER | r | d | nd | νd |
|---|---|---|---|---|
| 1 | ∞ | 0.2000 | 1.88300 | 40.76 |
| 2 | 1.014 | 0.4525 | | |
| 3 | ∞ | 0.3000 | 1.48749 | 70.23 |
| 4 | 1.398 | 0.2646 | | |
| 5 | ∞ | 0.3000 | 1.52100 | 65.12 |
| 6 | ∞ | D6 | | |
| 7* | 1.0896 | 0.4500 | 1.80610 | 40.92 |
| 8 | 1.138 | D8 | | |
| 6 | AS | 0.0300 | | |
| 10 | 3.175 | 0.6000 | 1.80100 | 34.97 |
| 11 | −4.098 | 0.7294 | | |
| 12 | 2.588 | 0.9000 | 1.81600 | 46.62 |
| 13 | −1.020 | 0.2700 | 1.92286 | 18.90 |
| 14 | −19.273 | 0.8050 | | |
| 15 | 1.727 | 0.6600 | 1.51633 | 64.14 |
| 16 | ∞ | 0.3500 | 1.51000 | 62.01 |
| 17 | ∞ | 0.0000 | | |

TABLE 17
NON-SPHERICAL DATA

| SURFACE | | |
|---|---|---|
| NUMBER | R | k |
| 7 | 1.0896 | 0.0000 |

| A4 | A6 | A8 | A10 |
|---|---|---|---|
| −4.84012 × 10⁻² | −2.97882 × 10⁻² | 6.88672 × 10⁻¹ | −2.22298 × 10⁰ |

TABLE 18
ZOOM DATA

| | FAR OBJECT | NEAR OBJECT |
|---|---|---|
| | POINT | POINT |
| OBJECT DISTANCE | 10.500 | 3.000 |
| FOCAL LENGTH | 0.5148 | 0.5192 |
| F NUMBER | 3.666 | 3.659 |
| ANGLE OF VIEW (2ω) | 142.89 | 138.19 |
| IMAGE HEIGHT | 0.500 | 0.500 |
| OVERALL LENGTH | 7.812 | 7.812 |
| D6 | 0.5800 | 0.8000 |
| D8 | 0.9200 | 0.7000 |

TABLE 19
FOCAL LENGTH OF EACH LENS GROUP

| | |
|---|---|
| FIRST LENS GROUP | −0.7013 |
| SECOND LENS GROUP | 6.1245 |
| THIRD LENS GROUP | 1.9282 |

TABLE 20
CORRESPONDING VALUES OF
CONDITIONAL EXPRESSIONS

| | | |
|---|---|---|
| CONDITIONAL EXPRESSION (1) | L1_Rr/L2_Rr | 0.725 |
| CONDITIONAL EXPRESSION (2) | L1/L2 | 0.399 |
| CONDITIONAL EXPRESSION (3) | L2_SF | 1.000 |
| CONDITIONAL EXPRESSION (5) | L1/f2 | −0.186 |
| CONDITIONAL EXPRESSION (6) | L1/fw | −2.218 |
| CONDITIONAL EXPRESSION (7) | f1/fw | −1.362 |
| CONDITIONAL EXPRESSION (8) | thi_3g_L1/thi_3g_air | 0.823 |
| CONDITIONAL EXPRESSION (9) | v/fw | 0.427 |
| CONDITIONAL EXPRESSION (10) | G3_L1_SF | −0.127 |
| CONDITIONAL EXPRESSION (12) | G3_Lce_SF | −0.763 |

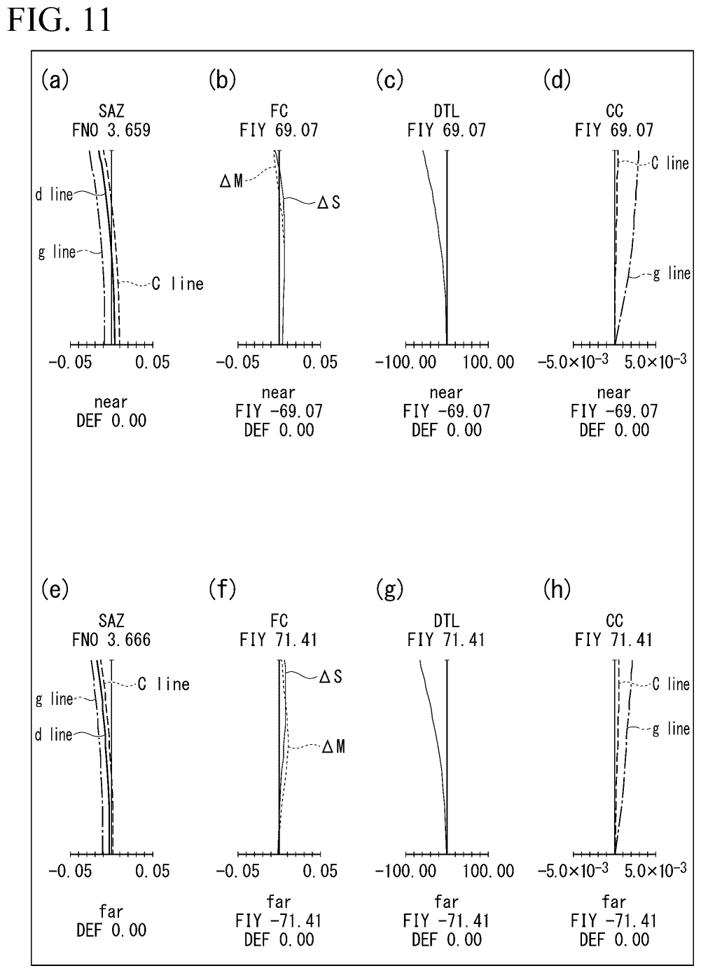

As can be seen from FIG. 11, the objective optical system of Example 4 also corrects the aberrations, including spherical surface aberration, non-point aberration, distortion aberration and magnification color aberration, and provides good aberration characteristics over the visible wavelength range.

### Example 5

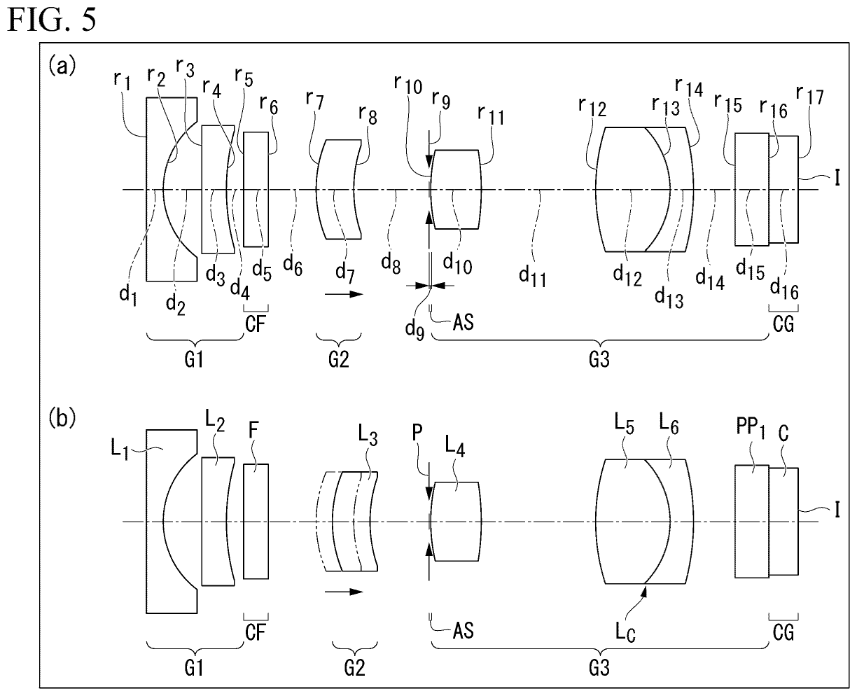

As shown in FIG. 5, in Example 5, the first lens group G1 is constituted by the plano-concave lens L₁ with negative refractive power and the plano-concave lens L₂ with negative refractive power, in order from the object side. The infrared filter F is disposed in a space on the image side of the plano-concave lens L₂.

The second lens group G2 is constituted by the single meniscus lens L₃ that is a positive meniscus lens.

The third lens group G3 is constituted by the biconvex lens L₄ with positive refractive power, the biconvex lens L₅ that is a positive lens, the meniscus lens L₆ that is a negative meniscus lens, and a parallel plate PP₁, in order from the object side. The aperture diaphragm P is disposed in a space on the object side of the biconvex lens L₄. The biconvex lens L₅ and the meniscus lens L₆ constitute the bonded lens L_{C}. The object side surface and the image side surface of the parallel plate PP1 are flat surfaces perpendicular to the optical axis. The cover glass C is disposed in a space on the image side of the third lens group G3. The image side surface of the parallel plate PP₁ and the object side surface of the cover glass C are in contact with each other.

Table 21 to Table 25 show numerical data of Example 5.

### [Numerical Example 5]

TABLE 21
SURFACE DATA

| SURFACE NUMBER | r | d | nd | νd |
|---|---|---|---|---|
| 1 | ∞ | 0.2000 | 1.88300 | 40.76 |
| 2 | 1.029 | 0.4543 | | |
| 3 | ∞ | 0.3000 | 1.88300 | 40.76 |
| 4 | 2.691 | 0.1993 | | |
| 5 | ∞ | 0.3000 | 1.52100 | 65.12 |
| 6 | ∞ | D6 | | |
| 7* | 1.4224 | 0.4500 | 1.80610 | 40.92 |
| 8 | 1.779 | D8 | | |
| 9 | AS | 0.0300 | | |
| 10 | 4.222 | 0.6000 | 1.72916 | 54.68 |
| 11 | −2.679 | 1.3742 | | |
| 12 | 2.450 | 0.9000 | 1.81600 | 46.62 |
| 13 | −1.020 | 0.2700 | 1.95906 | 17.47 |
| 14 | −3.111 | 0.5100 | | |
| 15 | ∞ | 0.4000 | 1.51633 | 64.14 |
| 16 | ∞ | 0.3500 | 1.51000 | 62.01 |
| 17 | ∞ | 0.0000 | | |

TABLE 22
NON-SPHERICAL DATA

| SURFACE | | |
|---|---|---|
| NUMBER | R | k |
| 7 | 1.4224 | 0.0000 |

| A4 | A6 | A8 | A10 |
|---|---|---|---|
| −6.42415 × 10⁻² | 6.61428 × 10⁻² | 9.01125 × 10⁻¹ | −3.56888 × 10⁰ |

TABLE 23
ZOOM DATA

| | FAR OBJECT | NEAR OBJECT |
|---|---|---|
| | POINT | POINT |
| OBJECT DISTANCE | 10.500 | 3.000 |
| FOCAL LENGTH | 0.5139 | 0.5136 |
| F NUMBER | 3.682 | 3.680 |
| ANGLE OF VIEW (2ω) | 143.54 | 141.76 |
| IMAGE HEIGHT | 0.500 | 0.500 |
| OVERALL LENGTH | 7.808 | 7.808 |
| D6 | 0.5800 | 0.7700 |
| D8 | 0.8900 | 0.7000 |

TABLE 24
FOCAL LENGTH OF EACH LENS GROUP

| | |
|---|---|
| FIRST LENS GROUP | −0.7311 |
| SECOND LENS GROUP | 5.5917 |
| THIRD LENS GROUP | 1.8626 |

TABLE 25
CORRESPONDING VALUES OF
CONDITIONAL EXPRESSIONS

| | | |
|---|---|---|
| CONDITIONAL EXPRESSION (1) | L1_Rr/L2_Rr | 0.382 |
| CONDITIONAL EXPRESSION (2) | L1/L2 | 0.382 |
| CONDITIONAL EXPRESSION (3) | L2_SF | 1.001 |
| CONDITIONAL EXPRESSION (5) | L1/f2 | −0.207 |
| CONDITIONAL EXPRESSION (6) | L1/fw | −2.254 |
| CONDITIONAL EXPRESSION (7) | f1/fw | −1.423 |
| CONDITIONAL EXPRESSION (8) | thi_3g_L1/thi_3g_air | 0.437 |
| CONDITIONAL EXPRESSION (9) | v/fw | 0.370 |
| CONDITIONAL EXPRESSION (10) | G3_L1_SF | 0.224 |
| CONDITIONAL EXPRESSION (12) | G3_Lce_SF | −0.119 |

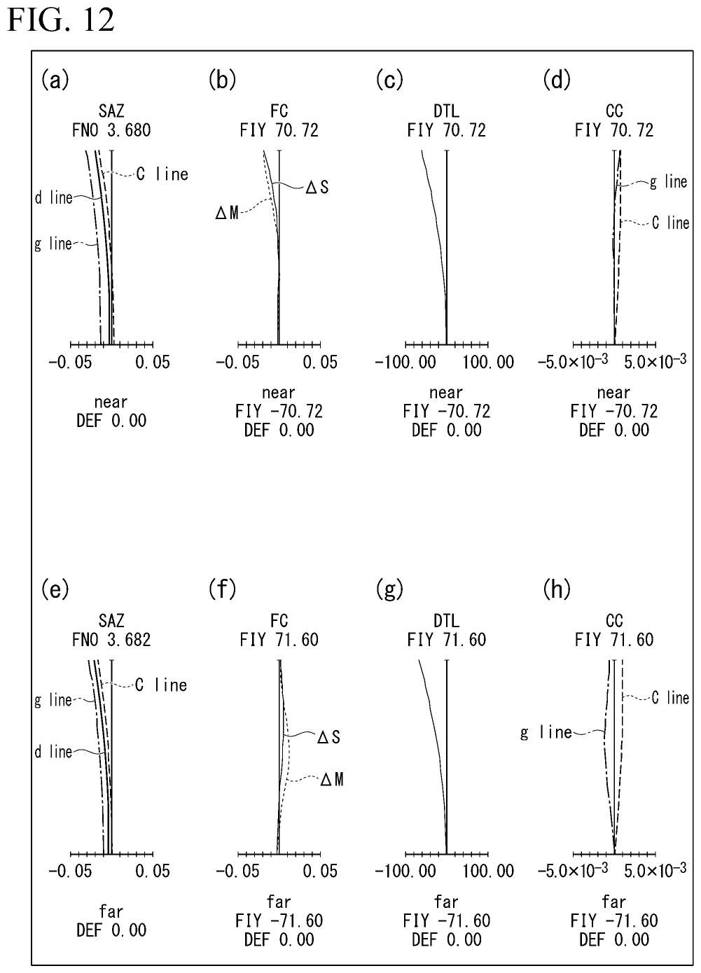

As can be seen from FIG. 12, the objective optical system of Example 5 also corrects the aberrations, including spherical surface aberration, non-point aberration, distortion aberration and magnification color aberration, and provides good aberration characteristics over the visible wavelength range.

### Example 6

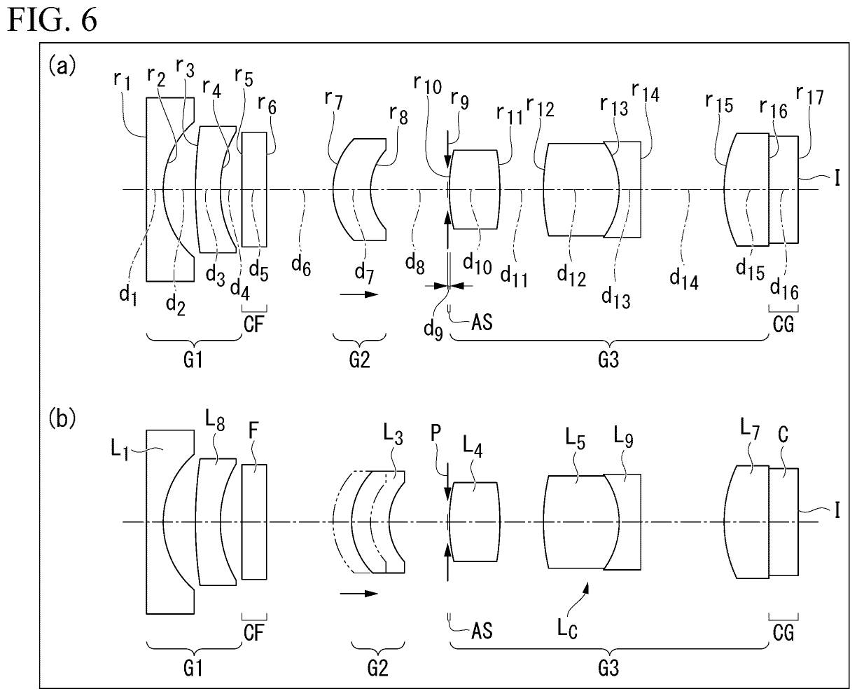

As shown in FIG. 6, in Example 6, the first lens group G1 is constituted by the plano-concave lens L₁ with negative refractive power and a meniscus lens L₈ with negative refractive power, in order from the object side. The meniscus lens L₈ is a negative meniscus lens, and corresponds to “a second lens” disclosed in the following claims. The object side surface of the meniscus lens L₈ is a convex surface protruding to the object side. The image side surface of the meniscus lens L₈ is a concave surface recessed to the object side. The infrared filter F is disposed in a space on the image side of the plano-concave lens L₂.

The second lens group G2 is the single meniscus lens L₃ that is a positive meniscus lens.

The third lens group G3 is constituted by the biconvex lens L₄ with positive refractive power, the biconvex lens L₅ that is a positive lens, a biconcave lens L₉ that is a negative meniscus lens, and the plano-convex lens L₇ with positive refractive power, in order from the object side. The aperture diaphragm P is disposed in a space on the object side of the biconvex lens L₄. The object side surface of the biconcave lens L₉ is a concave surface recessed to the image side. The image side surface of the biconcave lens L₉ is a concave surface recessed to the object side. The biconvex lens L₅ and the biconcave lens L₉ constitute the bonded lens L_{C}. That is, the image side surface of the biconvex lens L₅ and the object side surface of the biconcave lens L₉ are in contact with each other. The cover glass C is disposed in a space on the image side of the third lens group G3.

Table 26 to Table 30 show numerical data of Example 6.

### [Numerical Example 6]

TABLE 26
SURFACE DATA

| SURFACE NUMBER | r | d | nd | νd |
|---|---|---|---|---|
| 1 | ∞ | 0.2000 | 1.88300 | 40.76 |
| 2 | 1.094 | 0.3900 | | |
| 3 | 7.784 | 0.3000 | 1.88300 | 40.76 |
| 4 | 1.439 | 0.2569 | | |
| 5 | ∞ | 0.3000 | 1.52100 | 65.12 |
| 6 | ∞ | D6 | | |
| 7* | 0.7909 | 0.4500 | 1.80610 | 40.92 |
| 8 | 0.725 | D8 | | |
| 9 | AS | 0.0300 | | |
| 10 | 3.153 | 0.6000 | 1.80100 | 34.97 |
| 11 | −3.446 | 0.5262 | | |
| 12 | 2.564 | 0.9000 | 1.81600 | 46.62 |
| 13 | −1.020 | 0.2700 | 1.92286 | 18.90 |
| 14 | 60.689 | 0.9950 | | |
| 15 | 1.536 | 0.5408 | 1.51633 | 64.14 |
| 16 | ∞ | 0.3500 | 1.51000 | 62.01 |
| 17 | ∞ | 0.0000 | | |

TABLE 27
NON-SPHERICAL DATA

| SURFACE | | |
|---|---|---|
| NUMBER | R | k |
| 7 | 0.7909 | 0.0000 |

| A4 | A6 | A8 | A10 |
|---|---|---|---|
| −9.22598 × 10⁻² | −2.99737 × 10⁻¹ | 1.01174 × 10⁰ | −2.43326 × 10⁰ |

TABLE 28
ZOOM DATA

| | FAR OBJECT | NEAR OBJECT |
|---|---|---|
| | POINT | POINT |
| OBJECT DISTANCE | 10.500 | 3.000 |
| FOCAL LENGTH | 0.5150 | 0.5249 |
| F NUMBER | 3.800 | 3.792 |
| ANGLE OF VIEW (2ω) | 142.82 | 134.64 |
| IMAGE HEIGHT | 0.500 | 0.500 |
| OVERALL LENGTH | 7.811 | 7.811 |
| D6 | 0.7825 | 1.0025 |
| D8 | 0.9200 | 0.7000 |

TABLE 29
FOCAL LENGTH OF EACH LENS GROUP

| | |
|---|---|
| FIRST LENS GROUP | −0.6500 |
| SECOND LENS GROUP | 5.2044 |
| THIRD LENS GROUP | 1.9687 |

TABLE 30
CORRESPONDING VALUES OF
CONDITIONAL EXPRESSIONS

| | | |
|---|---|---|
| CONDITIONAL EXPRESSION (1) | L1_Rr/L2_Rr | 0.760 |
| CONDITIONAL EXPRESSION (2) | L1/L2 | 0.606 |
| CONDITIONAL EXPRESSION (3) | L2 SF | 1.454 |
| CONDITIONAL EXPRESSION (5) | L1/f2 | −0.237 |
| CONDITIONAL EXPRESSION (6) | L1/fw | −2.393 |
| CONDITIONAL EXPRESSION (7) | f1/fw | −1.262 |
| CONDITIONAL EXPRESSION (8) | thi_3g_L1/thi_3g_air | 1.140 |
| CONDITIONAL EXPRESSION (9) | v/fw | 0.427 |
| CONDITIONAL EXPRESSION (10) | G3_L1_SF | −0.044 |
| CONDITIONAL EXPRESSION (12) | G3_Lce_SF | −1.088 |

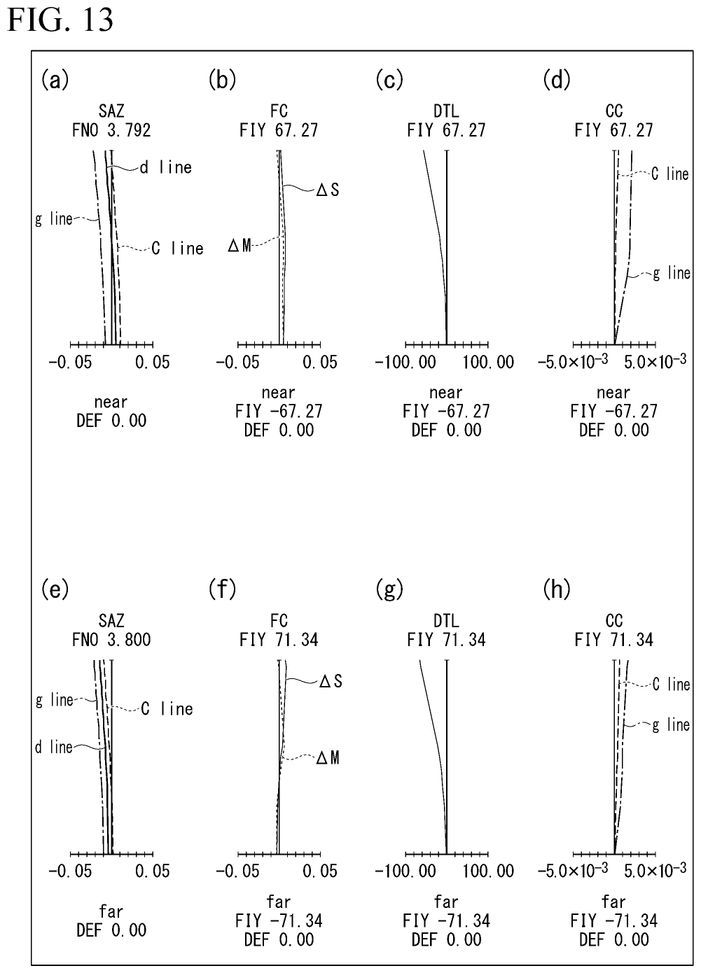

As can be seen from FIG. 13, the objective optical system of Example 6 also corrects the aberrations, including spherical surface aberration, non-point aberration, distortion aberration and magnification color aberration, and provides good aberration characteristics over the visible wavelength range.

### Example 7

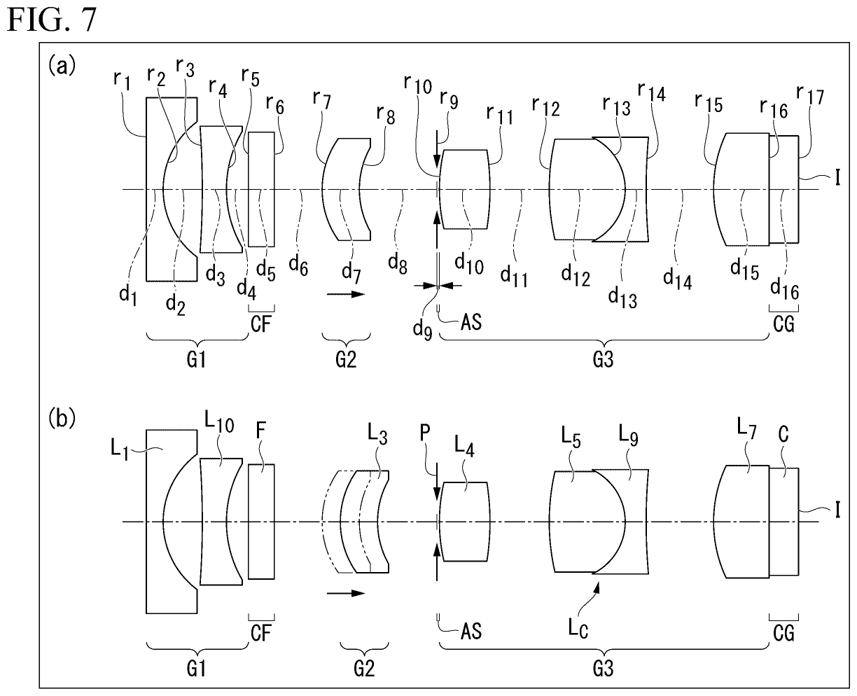

As shown in FIG. 7, in Example 7, the first lens group G1 is constituted by the plano-concave lens L₁ with negative refractive power and a biconcave lens L₁₀ with negative refractive power, in order from the object side. The biconcave lens L₁₀ corresponds to “a second lens” disclosed in the following claims. The object side surface of the biconcave lens L₁₀ is a concave surface recessed to the image side. The image side surface of the biconcave lens L₁₀ is a concave surface recessed to the object side. The infrared filter F is disposed in a space on the image side of the biconcave lens L₁₀.

The second lens group G2 is constituted by the single meniscus lens L₃ that is a positive meniscus lens.

The third lens group G3 is constituted by the biconvex lens L₄ with positive refractive power, the biconvex lens L₅ that is a positive lens, the biconcave lens L₉ that is a negative meniscus lens, and the plano-convex lens L₇ with positive refractive power, in order from the object side. The aperture diaphragm P is disposed in a space on the object side of the biconvex lens L₄. The object side surface of the biconcave lens L₉ is a concave surface recessed to the image side. The image side surface of the biconcave lens L₉ is a concave surface recessed to the object side. The biconvex lens L₅ and the biconcave lens L₉ constitute the bonded lens L_{C}. That is, the image side surface of the biconvex lens L₅ and the object side surface of the biconcave lens L₉ are in contact with each other. The cover glass C is disposed in a space on the image side of the third lens group G3.

Table 31 to Table 35 show numerical data of Example 7.

### [Numerical Example 7]

TABLE 31
SURFACE DATA

| SURFACE NUMBER | r | d | nd | νd |
|---|---|---|---|---|
| 1 | ∞ | 0.2000 | 1.88300 | 40.76 |
| 2 | 1.036 | 0.4544 | | |
| 3 | −23.091 | 0.3000 | 1.48749 | 70.23 |
| 4 | 1.368 | 0.2665 | | |
| 5 | ∞ | 0.3000 | 1.52100 | 65.12 |
| 6 | ∞ | D6 | | |
| 7* | 1.0388 | 0.4500 | 1.80610 | 40.92 |
| 8 | 1.065 | D8 | | |
| 9 | AS | 0.0300 | | |
| 10 | 3.176 | 0.6000 | 1.80100 | 34.97 |
| 11 | −3.932 | 0.7268 | | |
| 12 | 2.611 | 0.9000 | 1.81600 | 46.62 |
| 13 | −0.724 | 0.2700 | 1.84666 | 23.78 |
| 14 | 25.665 | 0.8000 | | |
| 15 | 1.646 | 0.6600 | 1.51633 | 64.14 |
| 16 | ∞ | 0.3500 | 1.51000 | 62.01 |
| 17 | ∞ | | | |

TABLE 32
NON-SPHERICAL DATA

| SURFACE | | |
|---|---|---|
| NUMBER | R | k |
| 7 | 1.0388 | 0.0000 |

| A4 | A6 | A8 | A10 |
|---|---|---|---|
| −5.53364 × 10⁻² | −8.30159 × 10⁻² | 8.61031 × 10⁻¹ | −2.36864 × 10⁰ |

TABLE 33
ZOOM DATA

| | FAR OBJECT | NEAR OBJECT |
|---|---|---|
| | POINT | POINT |
| OBJECT DISTANCE | 10.500 | 3.000 |
| FOCAL LENGTH | 0.5151 | 0.5203 |
| F NUMBER | 3.689 | 3.682 |
| ANGLE OF VIEW (2ω) | 142.75 | 137.48 |
| IMAGE HEIGHT | 0.500 | 0.500 |
| OVERALL LENGTH | 7.808 | 7.808 |
| D6 | 0.5800 | 0.8000 |
| D8 | 0.9200 | 0.7000 |

TABLE 34
FOCAL LENGTH OF EACH LENS GROUP

| | |
|---|---|
| FIRST LENS GROUP | −0.6908 |
| SECOND LENS GROUP | 5.9970 |
| THIRD LENS GROUP | 1.9368 |

TABLE 35
CORRESPONDING VALUES OF
CONDITIONAL EXPRESSIONS

| | | |
|---|---|---|
| CONDITIONAL EXPRESSION (1) | L1_Rr/L2_Rr | 0.757 |
| CONDITIONAL EXPRESSION (2) | L1/L2 | 0.444 |
| CONDITIONAL EXPRESSION (3) | L2_SF | 0.888 |
| CONDITIONAL EXPRESSION (5) | L1/f2 | −0.195 |
| CONDITIONAL EXPRESSION (6) | L1/fw | −2.265 |
| CONDITIONAL EXPRESSION (7) | f1/fw | −1.341 |
| CONDITIONAL EXPRESSION (8) | thi_3g_L1/thi_3g_air | 0.826 |
| CONDITIONAL EXPRESSION (9) | v/fw | 0.427 |
| CONDITIONAL EXPRESSION (10) | G3_L1_SF | −0.106 |
| CONDITIONAL EXPRESSION (12) | G3_Lce_SF | −1.226 |

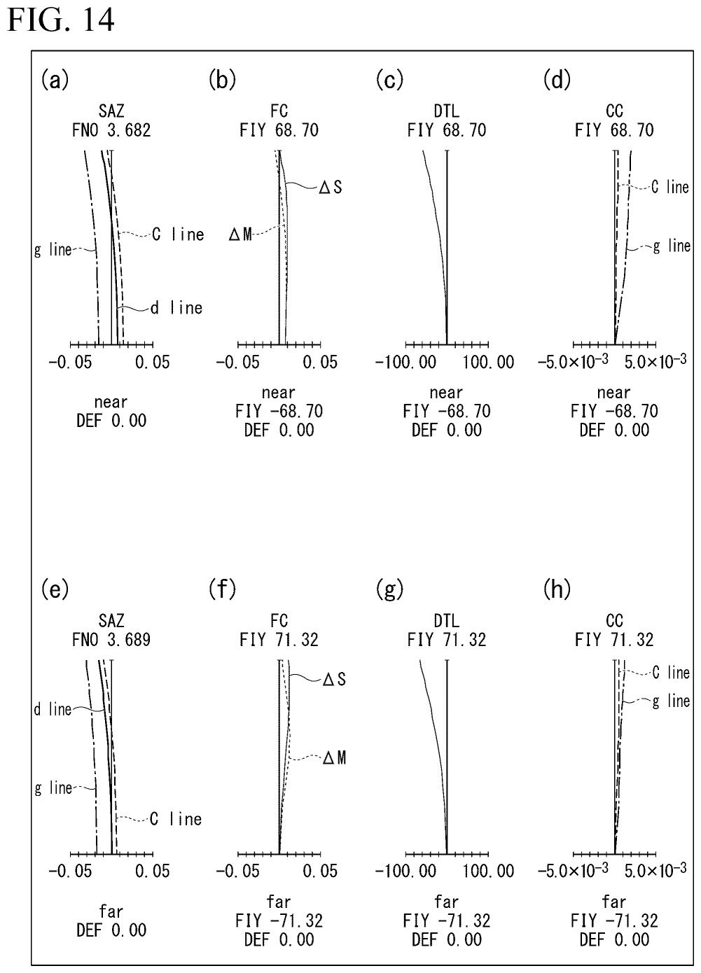

As can be seen from FIG. 14, the objective optical system of Example 7 also corrects the aberrations, including spherical surface aberration, non-point aberration, distortion aberration and magnification color aberration, and provides good aberration characteristics over the visible wavelength range.

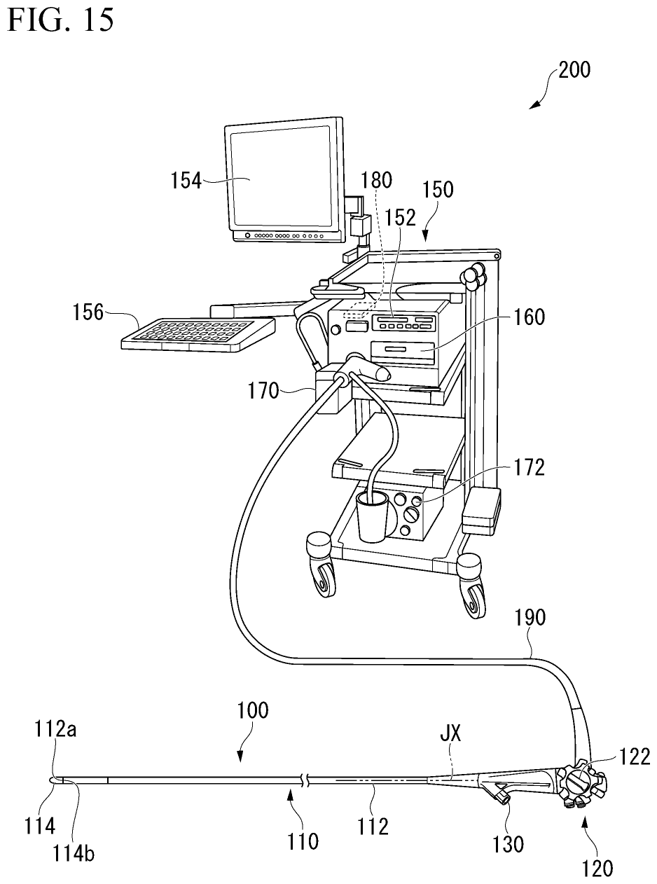

Next, an endoscope and an imaging device of the embodiment will be described. FIG. 15 is a schematic view of an endoscope 100 and an imaging device 200 of the embodiment.

As shown in FIG. 15, the endoscope 100 includes an insertion part 110, and an operation part 120. The insertion part 110 is elongated and configured so as to be insertable into a patient's body cavity (not shown). The insertion part 110 has an extension portion 112, and a tip portion 114. The extension portion 112 can be freely bent along an axis JX by a user's operation using the operation part 120 (not shown). That is, the axis shape of the extension portion 112 along the axis JX can be freely changed according to the anatomical passages into which it is inserted, such as the stomach, duodenum, kidney, ureter, etc. The extension portion 112 is formed of a flexible material. The tip portion 114 is disposed on a tip 112a of the extension portion 112, has substantially the same diameter as that of the extension portion 112, and is inserted through the anatomical passages integrally with the extension portion 112. That is, the tip 112a of the extension portion 112 is connected to a base end 114b of the tip portion 114.

While not shown, the insertion part 110 includes, for example, a plurality of extremely elongated functional members such as a treatment tool such as a cholangioscope, a light guide cable, an electrical cable, a fluid passage, a guide wire, and a pull wire, and a covering member that covers these functional members from an outer circumference in a radial direction of the axis JX. The objective optical system of the embodiment is accommodated in the tip portion 114 of the insertion part 110.

The operation part 120 is connected to a base end of the extension portion 112 of the insertion part 110. That is, the operation part 120 is connected to the base end opposite to the tip 112a connected to the tip portion 114 in the extension portion 112. The operation part 120 has a control knob 122, and a port 130. The control knob 122 is used by the user to manually advance or retract the insertion part 110, change the axis shape of the extension portion 112 to bend it, or change the direction in which the tip portion 114 faces. The control knob 122 corresponds to “a handle” disclosed in the following claims. The port 130 is configured to allow various types of functional members, such as electrical cables, guide wires, auxiliary scopes, fluid tubes, etc., to be attached to the operation part 120 for connection with the insertion part 110.

The imaging device 200 includes the endoscope 100, and a control device 150. The control device 150 has a controller 152, an output device 154, an input device 156, a light source 160, a fluid source 170, and a suction pump 172. The controller 152 receives data related to an observation object from the endoscope 100 and transmits data to the endoscope 100, and has an imaging element 180. The operation part 120 of the endoscope 100 is connected to the controller 152 via a connecting portion 190 such as a universal code or the like. The imaging element 180 receives an image acquired by the objective optical system of the embodiment, i.e., an image captured on the image plane I of the objective optical system, via the connecting portion 190. The imaging element 180 processes the received image, converts the processed image into an electrical signal, and transmits the electrical signal to the output device 154. The imaging element 180 is an image sensor such as a complementary metal-oxide semiconductor (CMOS), a charge coupled device (CCD), or the like.

The output device 154 outputs a plurality of pieces of information including images of the observation object or information related to the observation object transmitted from the imaging element 180, information transmitted from the controller 152, and information related to the operation of the endoscope 100. The output device 154, for example, as described above, is a display capable of displaying the plurality of pieces of information transmitted to the output device 154. The input device 156 mainly inputs a plurality of pieces of information, including an operation of the endoscope 100 and information regarding a testee, to the controller 152. The output device 154 is, for example, a keyboard, but may be a mouse or the like.

The light source 160 emits light in order to acquire an image of the observation object. The light emit emitted from the light source 160 is radiated from the tip portion 114 toward an observation target portion via a fiber link, a light guide cable inserted through the connecting portion 190, the operation part 120, and the insertion part 110 of the endoscope 100, and the like. The fluid source 170 is configured to be able to communicate with the controller 152 and supplies liquids such as air and treated water to the endoscope 100 via the port 130. The suction pump 172 evacuates a fluid from the anatomical region into which the insertion part 110 of the endoscope 100 is inserted, and has a port for, for example, generating vacuum suction.

The endoscope 100 and the imaging device 200 of the above-mentioned embodiment include the objective optical system of the embodiment. For this reason, according to the endoscope 100 and the imaging device 200 of the embodiment, a miniaturized tip portion 114 and a small diameter extension portion 112 of the endoscope 100 can be achieved, a high-performance objective optical system can be used to observe observation objects such as affected areas with high resolution, and the imaging element 180 can be used to obtain high-resolution images of the observation objects.

Further, the endoscope 100 and the imaging device 200 as described above are examples of the endoscope and the imaging device of the embodiment. Accordingly, the configuration of the endoscope and the imaging device of the embodiment may be changed as appropriate from the configurations of the endoscope 100 and the imaging device 200. For example, the operation part 120 of the endoscope 100 may accommodate a power source, a light source, an imaging element, and various other supply devices (not shown). In addition, for example, in the imaging device 200, the fluid source 170 and the suction pump 172 may be omitted, a videoscope (not shown) may be provided, and a storage device or a communication terminal (not shown) may be connected in a wired or wireless manner.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. An objective optical system constituted by a first lens group with negative refractive power, a second lens group with positive refractive power, and a third lens group with positive refractive power, in order from an object side,
wherein the second lens group is moved from the object side to an image side to perform focusing from a far object point to a near object point,
the first lens group is constituted by two lenses including a first lens that is a negative lens and a second lens that is a negative lens facing a concave surface on the image side,
the second lens group is constituted by a single positive meniscus lens facing a convex surface on the object side,
the third lens group has a single lens with positive refractive power and a bonded lens of a positive lens and a negative lens, in order from the object side, and
the following Conditional expressions (1) and (8) are satisfied:
$$0.01 < L1\_Rr / L2\_Rr < 0.95 \qquad ( 1 )$$
$$0.25 < thi\_ 3 g\_L1 / thi\_ 3 g\_air < 1.5 \qquad ( 8 )$$
here, L1_Rr: a radius of curvature of an image side surface of the first lens,
L2_Rr: a radius of curvature of an image side surface of the second lens,
thi_3g_L1: a thickness on an optical axis of the single lens, and
thi_3g_air: an air gap on the optical axis of the single lens and the bonded lens.

2. The objective optical system according to claim 1, wherein the following Conditional expression (2) is satisfied:
$$0.136 < L 1 / L 2 < 0 \text{.95} \qquad ( 2 )$$
here, L1: a focal length of the first lens, and
L2: a focal length of the second lens.

3. The objective optical system according to claim 1, wherein the following Conditional expression (3) is satisfied:
$$0.2 < L2\_SF < 1.85 \qquad ( 3 )$$
here, L2_SF: a shaping factor of the second lens, and
the shaping factor of the second lens is expressed by the following Equation (4):
$$L2\_SF = ( L2\_Lr - L2\_Rr ) / ( L2\_Lr + L2\_Rr ) \qquad ( 4 )$$
also
L2_Lr: a radius of curvature of an object side surface of the second lens, and
L2_Rr: a radius of curvature of an image side surface of the second lens.

4. The objective optical system according to claim 1, wherein the following Conditional expression (5) is satisfied:
$$- 0.4 < L 1 / f 2 < - 0 \text{.145} \qquad ( 5 )$$
here, L1: a focal length of the first lens, and
f2: a focal length of the second lens group.

5. The objective optical system according to claim 1, wherein the following Conditional expression (6) is satisfied:
$$- 3 < L 1 / fw < - 0.9 \; 55 \qquad ( 6 )$$
here, L1: a focal length of the first lens, and
fw: a focal length of the entire objective optical system when focusing to the far object point.

6. The objective optical system according to claim 1, wherein the following Conditional expression (7) is satisfied:
$$- 3 < f 1 / fw < - 1 \text{.06} \qquad ( 7 )$$
here, f1: a focal length of the first lens group, and
fw: a focal length of the entire objective optical system when focusing to the far object point.

7. The objective optical system according to claim 1, wherein the following Conditional expression (9) is satisfied:
$$0.325 < v / fw < 0.6 \qquad ( 9 )$$
here, v: an amount of movement of the second lens group from when focusing to the far object point to when focusing to the near object point, and
fw: a focal length of the entire objective optical system when focusing to the far object point.

8. The objective optical system according to claim 1, wherein the following Conditional expression (10) is satisfied:
$$- 0 \text{.4} < G3\_L 1 \_SF < 0.4 \qquad ( 10 )$$
here, G3_L1_SF: a shaping factor of the single lens, and
the shaping factor of the single lens is expressed by the following Equation (11),
$$G3\_L 1 \_SF = ( G3\_L 1 \_Lr + G3\_L 1 \_Rr ) / ( G3\_L 1 \_Lr - G3\_L 1 \_Rr ) , \qquad ( 11 )$$
also,
G3_L1_Lr: a radius of curvature of an object side surface of the single lens, and
G3_L1_Rr: a radius of curvature of an image side surface of the single lens.

9. The objective optical system according to claim 1, wherein the following Conditional expression (12) is satisfied:
$$- 1 \text{.5} < G3\_Lce \_SF < - 0.2 \qquad ( 12 )$$
here, G3_Lce_SF: a shaping factor of the bonded lens, and
the shaping factor of the bonded lens is expressed by the following Equation (13),
$$( 13 )$$
$$G3\_Lce \_SF = ( G3\_Lce \_Lr + G3\_Lce \_Rr ) / ( G3\_Lce \_Lr - G3\_Lce \_Rr )$$
also,
G3_Lce_Lr: a radius of curvature of an object side surface of the bonded lens, and
G3_Lce_Rr: a radius of curvature of an image side surface of the bonded lens.

10. An endoscope comprising:
a tip portion in which the objective optical system according to claim 1 is accommodated;
an extension portion connected to a base end of the tip portion to be bendable; and
an operation part connected to a base end opposite to a tip of the extension portion connected to the tip portion, and having a handle configured to change an axis shape of the extension portion.

11. An imaging device comprising:
the endoscope according to claim 10; and
an imaging element configured to convert an image acquired by the objective optical system into an electrical signal.
